SEQUENCE LISTING (66 sequences)

SEQ ID NO: 1 - DNA, 642 bp, Escherichia coli
atgaaaaactggaaaacaagtgcagaatcaatcctgaccaccggcccggttgtaccggttatcgtggtaaaaaaactggaacacgcggtgccgatggcaaaagcgttggttgctggtggggtgcgcgttctggaagtgactctgcgtaccgagtgtgcagttgacgctatccgtgctatcgccaaagaagtgcctgaagcgattgtgggtgccggtacggtgctgaatccacagcagctggcagaagtcactgaagcgggtgcacagttcgcaattagcccgggtctgaccgagccgctgctgaaagctgctaccgaagggactattcctctgattccggggatcagcactgtttccgaactgatgctgggtatggactacggtttgaaagagttcaaattcttcccggctgaagctaacggcggcgtgaaagccctgcaggcgatcgcgggtccgttctcccaggtccgtttctgcccgacgggtggtatttctccggctaactaccgtgactacctggcgctgaaaagcgtgctgtgcatcggtggttcctggctggttccggcagatgcgctggaagcgggcgattacgaccgcattactaagctggcgcgtgaagctgtagaaggcgctaagctgtaa

SEQ ID NO: 2 - DNA, 1812 bp, Escherichia coli
atgaatccacaattgttacgcgtaacaaatcgaatcattgaacgttcgcgcgagactcgctctgcttatctcgcccggatagaacaagcgaaaacttcgaccgttcatcgttcgcagttggcatgcggtaacctggcacacggtttcgctgcctgccagccagaagacaaagcctctttgaaaagcatgttgcgtaacaatatcgccatcatcacctcctataacgacatgctctccgcgcaccagccttatgaacactatccagaaatcattcgtaaagccctgcatgaagcgaatgcggttggtcaggttgcgggcggtgttccggcgatgtgtgatggtgtcacccaggggcaggatggaatggaattgtcgctgctaagccgcgaagtgatagcgatgtctgcggcggtggggctgtcccataacatgtttgatggtgctctgttcctcggtgtgtgcgacaagattgtcccgggtctgacgatggcagccctgtcgtttggtcatttgcctgcggtgtttgtgccgtctggaccgatggcaagcggtttgccaaataaagaaaaagtgcgtattcgccagctttatgccgaaggtaaagtggaccgcatggccttactggagtcagaagccgcgtcttaccatgcgccgggaacatgtactttctacggtactgccaacaccaaccagatggtggtggagtttatggggatgcagttgccaggctcttcttttgttcatccggattctccgctgcgcgatgctttgaccgccgcagctgcgcgtcaggttacacgcatgaccggtaatggtaatgaatggatgccgatcggtaagatgatcgatgagaaagtggtggtgaacggtatcgttgcactgctggcgaccggtggttccactaaccacaccatgcacctggtggcgatggcgcgcgcggccggtattcagattaactgggatgacttctctgacctttctgatgttgtaccgctgatggcacgtctctacccgaacggtccggccgatattaaccacttccaggcggcaggtggcgtaccggttctggtgcgtgaactgctcaaagcaggcctgctgcatgaagatgtcaatacggtggcaggttttggtctgtctcgttatacccttgaaccatggctgaataatggtgaactggactggcgggaaggggcggaaaaatcactcgacagcaatgtgatcgcttccttcgaacaacctttctctcatcatggtgggacaaaagtgttaagcggtaacctgggccgtgcggttatgaaaacctctgccgtgccggttgagaaccaggtgattgaagcgccagcggttgtttttgaaagccagcatgacgttatgccggcctttgaagcgggtttgctggaccgcgattgtgtcgttgttgtccgtcatcaggggccaaaagcgaacggaatgccagaattacataaactcatgccgccacttggtgtattattggaccggtgtttcaaaattgcgttagttaccgatggacgactctccggcgcttcaggtaaagtgccgtcagctatccacgtaacaccagaagcctacgatggcgggctgctggcaaaagtgcgcgacggggacatcattcgtgtgaatggacagacaggcgaactgacgctgctggtagacgaagcggaactggctgctcgcgaaccgcacattcctgacctgagcgcgtcacgcgtgggaacaggacgtgaattattcagcgccttgcgtgaaaaactgtccggtgccgaacagggcgcaacctgtatcactttttaa

SEQ ID NO: 3 - DNA, 1476 bp, Escherichia coli
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 4 - DNA, 1812 bp, Escherichia coli; identical to SEQ ID NO: 2

SEQ ID NO: 5 - DNA, 1827 bp, Klebsiella pneumoniae
atgccgttaatagccgggattgatatcggcaacgccaccaccgaggtggcgctggcgtccgatgacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttcagcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaataa

SEQ ID NO: 6 - DNA, 1671 bp, Klebsiella pneumoniae
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgagctggacggcaaacgccgggaccagtttgacatgatcgaccggtttatcgccgattacgcgatcaacgttgagcgcacagagcaggcaatgcgcctggaggcggtggaaatagcccgcatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctatgcgccgtttaacgccctggcgctgttggtcggctcgcagtgcggccgtcccggcgtgttgacgcagtgctcggtggaagaggccaccgagctggagctgggcatgcgtggcttaaccagctacgccgagacggtgtcggtctacggcactgaagcggtatttaccgacggcgatgatactccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattaccaaaggcgccggggttcaggggctgcaaaacggcgcagtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacattctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcgacagttcgaggtggtgagcgcggtcaacgacatcaatgactatcaggggccgggcaccggctatcgcatctctgccgaacgctgggcggagatcaaaaatattccgggcgtggttcagcctgacaccattgaataataa

SEQ ID NO: 7 - DNA, 588 bp, Klebsiella pneumoniae
atgcaacagacaactcaaattcagccctcttttaccctgaaaacccgcgagggcggggtagcttctgccgatgaacgtgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgccgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacgtcgacttagtaagggagtaataa

SEQ ID NO: 8 - DNA, 357 bp, Klebsiella pneumoniae
atgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtattgggctcagcgcgtccggcgagatagccctcactcatgcccagctgccggcggacgcgccgctggctaccggacacgtcaccgatagcgacgatcatctgcgtacgctcggcgccaacgccgggcagctggttaaagtcctgccgttaagtgagagaaactaataa

SEQ ID NO: 9 - DNA, 432 bp, Klebsiella pneumoniae
atgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaataaaaa

SEQ ID NO: 10 - DNA, 1416 bp, Bacillus cereus
atgaaaaacaaatggtataaaccgaaacggcattggaaggagatcgagttatggaaggacgttccggaagagaaatggaacgattggctttggcagctgacacacactgtaagaacgttagatgatttaaagaaagtcattaatctgaccgaggatgaagaggaaggcgtccgtatttctaccaaaacgatccccttaaatattacaccttactatgcttctttaatggaccccgacaatccgagatgcccggtacgcatgcagtctgtgccgctttctgaagaaatgcacaaaacaaaatacgatatggaagacccgcttcatgaggatgaagattcaccggtacccggtctgacacaccgctatcccgaccgtgtgctgtttcttgtcacgaatcaatgttccgtgtactgccgccactgcacacgccggcgcttttccggacaaatcggaatgggcgtccccaaaaaacagcttgatgctgcaattgcttatatccgggaaacacccgaaatccgcgattgtttactttcaggcggtgatgggctgctcatcaacgaccaaattttagaatatattttaaaagagctgcgcagcattccgcatctggaagtcatccgcatcggaacacgtgctcccgtcgtctttccgcagcgcattaccgatcatctgtgcgagatgttgaaaaaatatcatccggtctggctgaacacccattttaacacaagcatcgaaatgacagaagaatccgttgaggcatgtgaaaagctggtgaacgcgggagtgccggtcggaaatcaggctgtcgtattagcaggtattaatgattcggttccaattatgaaaaagctcatgcatgacttggtaaaaatcagagtccgtccttattatatttaccaatgtgatctgtcagaaggaataaggcatttccgtgctcctgtttccaaaggtttggagatcattgaagggctgagaggtcatacctcaggctatgcggttcctacctttgtcgttcacgcaccaggcggaggaggtaaaatcgccctgcagccgaactatgtcctgtcacaaagtcctgacaaagtgatcttaagaaattttgaaggtgtgattacgtcatatccggaaccagagaattatatccccaatcaggcagacgcctattttgagtccgttttccctgaaaccgctgacaaaaaggagccgatcgggctgagtgccctttttgctgacaaagaagtttcgtctacacctgaaaatgtagacagaatcaaacggcgtgaggcatacatcgcaaatccggagcatgaaacattaaaagatcggcgtgagaaaagaggtcagctcaaagaaaagaaatttttggcgcagcagaaaaaacagaaagagactgaatgcggaggggattcttcataa

SEQ ID NO: 11 - DNA, 879 bp, Bacillus cereus
atggaacataaaactttatcaataggtttcattggtattggcgtaatgggaaaaagtatggtttatcacttaatgcaagatggtcataaagtatatgtatataatagaacgaaagcaaaaacagattctttagtgcaagatggtgcacaatggtgtgatacgccaaaagagttagtgaagcaagttgatattgtaatgacaatggttggatatccacatgatgtagaagaagtgtattttggtatagaaggaattatagaacatgcaaaagaaggtacgatagcaattgactttacgacatctacacctactttagcaaaacgtattaatgaagttgcaaaaagcaaaaatatatatacgttagatgcacctgtctcaggaggagatgttggtgcgaaagaagcaaaactcgcaattatggtaggtggagagaaagaaatatatgatagatgcttacctttacttgaaaagttaggaacaaacattcaattacaaggaccagctgggagtggacaacatacaaaaatgtgcaatcaaattgcgattgcttccaatatgattggagtatgtgaggctgttgcttacgcgaagaaggctggattgaatccagataaagtgttagagagtatttcaacaggggcagcaggtagttggtcattaagtaatttagctcctcgaatgttaaaaggagactttgagccaggattttatgtaaagcattttatgaaagatatgaagattgctttagaggaagcagaaaaattacaattaccagtcccaggcttaagtttggcgaaagaattgtatgaagagttaattaaggatggcgaagaaaatagtggaacacaagtattatataaaaaatatataagggggtaa

SEQ ID NO: 12 - DNA, 1346 bp, Escherichia coli
atgaaccagccgctcaacgtggccccgccggtttccagcgaactcaacctgcgcgcccactggatgcccttctccgccaaccgcaacttccagaaggacccgcggatcatcgtcgccgccgaaggcagctggctgaccgacgacaagggccgcaaggtctacgacagcctgtccggcctgtggacctgcggcgccggccactcgcgcaaggaaatccaggaggcggtggctcgccagctcggcaccctcgactactcgccgggcttccagtacggccatccgctgtccttccagttggccgagaagatcgccgggttgctgccaggcgaactgaaccacgtgttcttcaccggttccggctccgagtgcgccgacacctcgatcaagatggcccgcgcctactggcgcctgaaaggccagccgcagaagaccaagctgatcggccgcgcccgcggctaccacggggtcaacgtcgccggcaccagcctcggcgggatcggtggcaaccgcaagatgttcggccagctgatggacgtcgaccatctgccgcacacccttcaaccgggcatggcgttcacccgcgggatggcccagaccggcggcgtcgagctggccaacgagctgctcaagctgatcgaactgcacgacgcctcgaacatcgccgcggtgatcgtcgagccgatgtccggctccgccggcgtactggtaccgccggtcggctacctgcagcgcctgcgcgagatctgcgaccagcacaacatcctgctgatcttcgacgaggtgatcaccgccttcggccgcctgggcacctacagcggcgccgagtacttcggcgtcaccccggacctgatgaacgtcgccaagcaggtcaccaacggcgccgtgccgatgggcgcggtgatcgccagcagcgagatctacgacaccttcatgaaccaggcgctgcccgagcacgcggtggagttcagccacggctacacctactccgcgcacccggtcgcctgcgccgccggcctcgccgcgctggacatcctggccagggacaacctggtgcagcagtccgccgagctggcgccgcacttcgagaagggcctgcacggcctgcaaggcgcgaagaacgtcatcgacatccgcaactgcggcctggccggcgcgatccagatcgccccgcgcgacggcgatccgaccgtgcgtccgttcgaggccggcatgaagctctggcaacagggtttctacgtgcgcttcggcggcgataccctgcaattcggcccgaccttcaacgccaggccggaagagctggaccgcctgttcgacgcggtcggcgaagcgctcaacggcatcgcctg

SEQ ID NO: 13 - DNA, 990 bp, Escherichia coli
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 14 - DNA, 1656 bp, Escherichia coli
atgaatctctggcaacaaaactacgatcccgccgggaatatctggctttccagtctgatagcatcgcttcccatcctgtttttcttctttgcgctgattaagctcaaactgaaaggatacgtcgccgcctcgtggacggtggcaatcgcccttgccgtggctttgctgttctataaaatgccggtcgctaacgcgctggcctcggtggtttatggtttcttctacgggttgtggcccatcgcgtggatcattattgcagcggtgttcgtctataagatctcggtgaaaaccgggcagtttgacatcattcgctcgtctattctttcgataacccctgaccagcgtctgcaaatgctgatcgtcggtttctgtttcggcgcgttccttgaaggagccgcaggctttggcgcaccggtagcaattaccgccgcattgctggtcggcctgggttttaaaccgctgtacgccgccgggctgtgcctgattgttaacaccgcgccagtggcatttggtgcgatgggcattccaatcctggttgccggacaggtaacaggtatcgacagctttgagattggtcagatggtggggcggcagctaccgtttatgaccattatcgtgctgttctggatcatggcgattatggacggctggcgcggtatcaaagagacgtggcctgcggtcgtggttgcgggcggctcgtttgccatcgctcagtaccttagctctaacttcattgggccggagctgccggacattatctcttcgctggtatcactgctctgcctgacgctgttcctcaaacgctggcagccagtgcgtgtattccgttttggtgatttgggggcgtcacaggttgatatgacgctggcccacaccggttacactgcgggtcaggtgttacgtgcctggacaccgttcctgttcctgacagctaccgtaacactgtggagtatcccgccgtttaaagccctgttcgcatcgggtggcgcgctgtatgagtgggtgatcaatattccggtgccgtacctcgataaactggttgcccgtatgccgccagtggtcagcgaggctacagcctatgccgccgtgtttaagtttgactggttctctgccaccggcaccgccattctgtttgctgcactgctctcgattgtctggctgaagatgaaaccgtctgacgctatcagcaccttcggcagcacgctgaaagaactggctctgcccatctactccatcggtatggtgctggcattcgcctttatttcgaactattccggactgtcatcaacactggcgctggcactggcgcacaccggtcatgcattcaccttcttctcgccgttcctcggctggctgggggtattcctgaccgggtcggatacctcatctaacgccctgttcgccgcgctgcaagccaccgcagcacaacaaattggcgtctctgatctgttgctggttgccgccaataccaccggtggcgtcaccggtaagatgatctccccgcaatctatcgctatcgcctgtgcggcggtaggcctggtgggcaaagagtctgatttgttccgctttactgtcaaacacagcctgatcttcacctgtatagtgggcgtgatcaccacgcttcaggcttatgtcttaacgtggatgattccttaa

SEQ ID NO: 15 - DNA, 1401 bp, Escherichia coli
atgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaa

SEQ ID NO: 16 - DNA, 1179 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattaataa

SEQ ID NO: 17 - DNA, 756 bp, Saccharomyces cerevisiae
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaataa

SEQ ID NO: 18 - DNA, 771 bp, Escherichia coli
atgcgacatcctttagtgatgggtaactggaaactgaacggcagccgccacatggttcacgagctggtttctaacctgcgtaaagagctggcaggtgttgctggctgtgcggttgcaatcgcaccaccggaaatgtatatcgatatggcgaagcgcgaagctgaaggcagccacatcatgctgggtgcgcaaaacgtggacctgaacctgtccggcgcattcaccggtgaaacctctgctgctatgctgaaagacatcggcgcacagtacatcatcatcggtcactctgaacgtcgtacttaccacaaagaatctgacgaactgatcgcgaaaaaattcgcggtgctgaaagagcagggcctgactccggttctgtgcatcggtgaaaccgaagctgaaaatgaagcgggcaaaactgaagaagtttgcgcacgtcagatcgacgcggtactgaaaactcagggtgctgcggcattcgaaggtgcggttatcgcttacgaacctgtatgggcaatcggtactggcaaatctgcaactccggctcaggcacaggctgttcacaaattcatccgtgaccacatcgctaaagttgacgctaacatcgctgaacaagtgatcattcagtacggcggctctgtaaacgcgtctaacgctgcagaactgtttgctcagccggatatcgacggcgcgctggttggtggtgcttctctgaaagctgacgccttcgcagtaatcgttaaagctgcagaagcggctaaacaggcttaataa

SEQ ID NO: 19 - DNA, 261 bp, Escherichia coli
atgttccagcaagaagttaccattaccgctccgaacggtctgcacacccgccctgctgcccagtttgtaaaagaagctaagggcttcacttctgaaattactgtgacttccaacggcaaaagcgccagcgcgaaaagcctgtttaaactgcagactctgggcctgactcaaggtaccgttgtgactatctccgcagaaggcgaagacgagcagaaagcggttgaacatctggttaaactgatggcggaactcgagtaataa

SEQ ID NO: 20 - DNA, 513 bp, Escherichia coli
atgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaataa

SEQ ID NO: 21 - DNA, 1437 bp, Escherichia coli
atgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaataa

SEQ ID NO: 22 - DNA, 1731 bp, Escherichia coli
atgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaataa

SEQ ID NO: 23 - DNA, 1533 bp, Escherichia coli
atgcgaattggcataccaagagaacggttaaccaatgaaacccgtgttgcagcaacgccaaaaacagtggaacagctgctgaaactgggttttaccgtcgcggtagagagcggcgcgggtcaactggcaagttttgacgataaagcgtttgtgcaagcgggcgctgaaattgtagaagggaatagcgtctggcagtcagagatcattctgaaggtcaatgcgccgttagatgatgaaattgcgttactgaatcctgggacaacgctggtgagttttatctggcctgcgcagaatccggaattaatgcaaaaacttgcggaacgtaacgtgaccgtgatggcgatggactctgtgccgcgtatctcacgcgcacaatcgctggacgcactaagctcgatggcgaacatcgccggttatcgcgccattgttgaagcggcacatgaatttgggcgcttctttaccgggcaaattactgcggccgggaaagtgccaccggcaaaagtgatggtgattggtgcgggtgttgcaggtctggccgccattggcgcagcaaacagtctcggcgcgattgtgcgtgcattcgacacccgcccggaagtgaaagaacaagttcaaagtatgggcgcggaattcctcgagctggattttaaagaggaagctggcagcggcgatggctatgccaaagtgatgtcggacgcgttcatcaaagcggaaatggaactctttgccgcccaggcaaaagaggtcgatatcattgtcaccaccgcgcttattccaggcaaaccagcgccgaagctaattacccgtgaaatggttgactccatgaaggcgggcagtgtgattgtcgacctggcagcccaaaacggcggcaactgtgaatacaccgtgccgggtgaaatcttcactacggaaaatggtgtcaaagtgattggttataccgatcttccgggccgtctgccgacgcaatcctcacagctttacggcacaaacctcgttaatctgctgaaactgttgtgcaaagagaaagacggcaatatcactgttgattttgatgatgtggtgattcgcggcgtgaccgtgatccgtgcgggcgaaattacctggccggcaccgccgattcaggtatcagctcagccgcaggcggcacaaaaagcggcaccggaagtgaaaactgaggaaaaatgtacctgctcaccgtggcgtaaatacgcgttgatggcgctggcaatcattctttttggctggatggcaagcgttgcgccgaaagaattccttgggcacttcaccgttttcgcgctggcctgcgttgtcggttattacgtggtgtggaatgtatcgcacgcgctgcatacaccgttgatgtcggtcaccaacgcgatttcagggattattgttgtcggagcactgttgcagattggccagggcggctgggttagcttccttagttttatcgcggtgcttatagccagcattaatattttcggtggcttcaccgtgactcagcgcatgctgaaaatgttccgcaaaaattaa

SEQ ID NO: 24 - DNA, 1020 bp, Escherichia coli
atgaaccaacgtaatgcttcaatgactgtgatcggtgccggctcgtacggcaccgctcttgccatcaccctggcaagaaatggccacgaggttgtcctctggggccatgaccctgaacatatcgcaacgcttgaacgcgaccgctgtaacgccgcgtttctccccgatgtgccttttcccgatacgctccatcttgaaagcgatctcgccactgcgctggcagccagccgtaatattctcgtcgtcgtacccagccatgtctttggtgaagtgctgcgccagattaaaccactgatgcgtcctgatgcgcgtctggtgtgggcgaccaaagggctggaagcggaaaccggacgtctgttacaggacgtggcgcgtgaggccttaggcgatcaaattccgctggcggttatctctggcccaacgtttgcgaaagaactggcggcaggtttaccgacagctatttcgctggcctcgaccgatcagacctttgccgatgatctccagcagctgctgcactgcggcaaaagtttccgcgtttacagcaatccggatttcattggcgtgcagcttggcggcgcggtgaaaaacgttattgccattggtgcggggatgtccgacggtatcggttttggtgcgaatgcgcgtacggcgctgatcacccgtgggctggctgaaatgtcgcgtcttggtgcggcgctgggtgccgaccctgccacctttatgggcatggcggggcttggcgatctggtgcttacctgtaccgacaaccagtcgcgtaaccgccgttttggcatgatgctcggtcagggcatggatgtacaaagcgcgcaggagaagattggtcaggtggtggaaggctaccgcaatacgaaagaagtccgcgaactggcgcatcgcttcggcgttgaaatgccaataaccgaggaaatttatcaagtattatattgcggaaaaaacgcgcgcgaggcagcattgactttactaggtcgtgcacgcaaggacgagcgcagcagccactaa

SEQ ID NO: 25 - DNA, 1533 bp, Escherichia coli; identical to SEQ ID NO: 23

SEQ ID NO: 26 - DNA, 966 bp, Escherichia coli
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaataa

SEQ ID NO: 27 - DNA, 1653 bp, Escherichia coli
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcttggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaataa

SEQ ID NO: 28 - DNA, 1083 bp, Escherichia coli
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaataa

SEQ ID NO: 29 - DNA, 966 bp, Escherichia coli
atgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgatcccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggagcgcgtgctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaagcctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaa

SEQ ID NO: 30 - DNA, 1395 bp, Escherichia coli
atgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 31 - DNA, 753 bp, Escherichia coli
atggctgtaactaagctggttctggttcgtcatggcgaaagtcagtggaacaaagaaaaccgtttcaccggttggtacgacgtggatctgtctgagaaaggcgtaagcgaagcaaaagcagcaggtaagctgctgaaagaggaaggttacagctttgactttgcttacacttctgtgctgaaacgcgctatccataccctgtggaatgtgctggacgaactggatcaggcatggctgcccgttgagaaatcctggaaactgaacgaacgtcactacggtgcgttgcagggtctgaacaaagcggaaactgctgaaaagtatggcgacgagcaggtgaaacagtggcgtcgtggttttgcagtgactccgccggaactgactaaagatgatgagcgttatccgggtcacgatccgcgttacgcgaaactgagcgagaaagaactgccgctgacggaaagcctggcgctgaccattgaccgcgtgatcccttactggaatgaaactattctgccgcgtatgaagagcggtgagcgcgtgatcatcgctgcacacggtaactctttacgtgcgctggtgaaatatcttgataacatgagcgaagaagagattcttgagcttaatatcccgactggcgtgccgctggtgtatgagttcgacgagaatttcaaaccgctgaaacgctattatctgggtaatgctgacgagatcgcagcgaaagcagcggcggttgcaaaccagggtaaagcgaagtaa

SEQ ID NO: 32 - DNA, 1299 bp, Escherichia coli
atgtccaaaatcgtaaaaatcatcggtcgtgaaatcatcgactcccgtggtaacccgactgttgaagccgaagtacatctggagggtggtttcgtcggtatggcagctgctccgtcaggtgcttctactggttcccgtgaagctctggaactgcgcgatggcgacaaatcccgtttcctgggtaaaggcgtaaccaaagctgttgctgcggtaaacggcccgatcgctcaggcgctgattggcaaagatgctaaagatcaggctggcattgacaagatcatgatcgacctggacggcaccgaaaacaaatccaaattcggcgcgaacgcaatcctggctgtatctctggctaacgccaaagctgctgcagctgctaaaggtatgccgctgtacgagcacatcgctgaactgaacggtactccgggcaaatactctatgccggttccgatgatgaacatcatcaacggtggtgagcacgctgacaacaacgttgatatccaggaattcatgattcagccggttggcgcgaaaactgtgaaagaagccatccgcatgggttctgaagttttccatcacctggcaaaagttctgaaagcgaaaggcatgaacactgctgttggtgacgaaggtggctatgcgccgaacctgggttccaacgctgaagctctggctgttatcgctgaagctgttaaagctgctggttatgaactgggcaaagacatcactttggcgatggactgcgcagcttctgaattctacaaagatggtaaatacgttctggctggcgaaggcaacaaagcgttcacctctgaagaattcactcacttcctggaagaactgaccaaacagtacccgatcgtttctatcgaagacggtctggacgaatctgactgggacggtttcgcataccagaccaaagttctgggcgacaaaatccagctggttggtgacgacctgttcgtaaccaacaccaagatcctgaaagaaggtatcgaaaaaggtatcgctaactccatcctgatcaaattcaaccagatcggttctctgaccgaaactctggctgcaatcaagatggcgaaagatgctggctacactgcagttatctctcaccgttctggcgaaactgaagacgctaccatcgctgacctggctgttggtactgctgcaggccagatcaaaactggttctatgagccgttctgaccgtgttgctaaatacaaccagctgattcgtatcgaagaagctctgggcgaaaaagcaccgtacaacggtcgtaaagagatcaaaggccaggcataa

SEQ ID NO: 33 - DNA, 1449 bp, Clostridium acetobutylicum
atgtttgaaaatatatcatcaaatggagtttataaaaatctatttgatggaaaatgggttgaaagtaagacaaataaaaccatagaaacgcattctccttatgatggaagtttaattggaaaagttcaggccttatcaaaagaggaagttgatgagatttttaaaagttcaagaacagctcagaaaaaatggggtgaaactccaataaatgagcgtgctagaatcatgcgtaaagcagctgatatactagatgataacgcagaatatatagcaaaaattctttcaaatgagatagcaaaagatttaaaatcttctctttcagaagtaaaaagaacagctgattttataagatttacagctaatgaaggtactcatatggaaggagaagctattaactcagataattttcctggttctaaaaaagataaactttctctagttgaaagagttcctttaggaatagttttagctatatctccttttaattatcctgtaaatctttctgggtctaaggttgctccagcacttatagctggaaatagtgttgttttaaaaccttctacaactggtgctataagcgcacttcatcttgcagaaatttttaatgcagctggtcttccagcaggtgttttaaacactgtaacaggaaaagggtctgaaataggcgattatttaattacccatgaagaagtaaactttattaactttacgggaagctctgctgtaggtaagcatatttcaaaaatagctggaatgatacctatggttcttgagcttggtggtaaagatgctgctatagttctcgaagatgccaatcttgaaacaacagctaaaagcatagtatctggagcatatggatactccggccaaaggtgtactgctgtaaaaagagttcttgtaatggataaagtagctgatgaattagttgaacttgttacaaaaaaagttaaagaattaaaggtaggtaatccttttgatgatgttacaataaccccacttatagacaacaaggcagcagattatgttcaaactctcattgacgacgctatcgaaaagggtgcaactcttatcgttggaaataagcgtaaagaaaatttaatgtatcctactttatttgataatgtaactgctgatatgcgtattgcttgggaagaaccatttggaccagttttacctattattcgtgtaaaaagcatggatgaagcaatagaattagcaaatagatctgaatatggtcttcaatctgcagtatttactgaaaatatgcatgatgccttttatattgccaataaattagatgttggaactgttcaagtaaataataagcctgaaagaggcccagatcacttcccattccttggaacaaagtcatcaggtatgggcactcaaggaattcgatacagtatagaggcaatgacaaggcataaatcaatagttttaaacctataa

SEQ ID NO: 34 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 35 - protein, 603 aa, Escherichia coli
MNPQLLRVTNRIIERSRETRSAYLARIEQAKTSTVHRSQLACGNLAHGFAACQPEDKASLKSMLRNNIAIITSYNDMLSAHQPYEHYPEIIRKALHEANAVGQVAGGVPAMCDGVTQGQDGMELSLLSREVIAMSAAVGLSHNMFDGALFLGVCDKIVPGLTMAALSFGHLPAVFVPSGPMASGLPNKEKVRIRQLYAEGKVDRMALLESEAASYHAPGTCTFYGTANTNQMVVEFMGMQLPGSSFVHPDSPLRDALTAAAARQVTRMTGNGNEWMPIGKMIDEKVVVNGIVALLATGGSTNHTMHLVAMARAAGIQINWDDFSDLSDVVPLMARLYPNGPADINHFQAAGGVPVLVRELLKAGLLHEDVNTVAGFGLSRYTLEPWLNNGELDWREGAEKSLDSNVIASFEQPFSHHGGTKVLSGNLGRAVMKTSAVPVENQVIEAPAVVFESQHDVMPAFEAGLLDRDCVVVVRHQGPKANGMPELHKLMPPLGVLLDRCFKIALVTDGRLSGASGKVPSAIHVTPEAYDGGLLAKVRDGDIIRVNGQTGELTLLVDEAELAAREPHIPDLSASRVGTGRELFSALREKLSGAEQGATCITF

SEQ ID NO: 36 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 37 - protein, 603 aa, Escherichia coli; identical to SEQ ID NO: 35

SEQ ID NO: 38 - protein, 607 aa, Klebsiella pneumoniae
MPLIAGIDIGNATTEVALASDDPQARAFVASGIVATTGMKGTRDNIAGTLAALEQALAKTPWSMSDVSRIYLNEAAPVIGDVAMETITETIITESTMIGHNPQTPGGVGVGVGTTIALGRLATLPAAQYAEGWIVLIDDAVDFLDAVWWLNEALDRGINVVAAILKKDDGVLVNNRLRKTLPVVDEVTLLEQVPEGVMAAVEVAAPGQVVRILSNPYGIATFFGLSPEETQAIVPIARALIGNRSAVVLKTPQGDVQSRVIPAGNLYISGEKRRGEADVAEGAEAIMQAMSACAPVRDIRGEPGTHAGGMLERVRKVMASLTGHEMSAIYIQDLLAVDTFIPRKVQGGMAGECAMENAVGMAAMVKADRLQMQVIARELSARLQTEVVVGGVEANMAIAGALTTPGCAAPLAILDLGAGSTDAAIVNAEGQITAVHLAGAGNMVSLLIKTELGLEDLSLAEAIKKYPLAKVESLFSIRHENGAVEFFREALSPAVFAKVVYIKEGELVPIDNASPLEKIRLVRRQAKEKVFVTNCLRALRQVSPGGSIRDIAFVVLVGGSSLDFEIPQLITEALSHYGVVAGQGNIRGTEGPRNAVATGLLLAGQAN

SEQ ID NO: 39 - protein, 555 aa, Klebsiella pneumoniae
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 40 - protein, 194 aa, Klebsiella pneumoniae
MQQTTQIQPSFTLKTREGGVASADERADEVVIGVGPAFDKHQHHTLIDMPHGAILKELIAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAEPVTLHVDLVRE

SEQ ID NO: 41 - protein, 117 aa, Klebsiella pneumoniae
MSLSPPGVRLFYDPRGHHAGAINELCWGLEEQGVPCQTITYDGGGDAAALGALAARSSPLRVGIGLSASGEIALTHAQLPADAPLATGHVTDSDDHLRTLGANAGQLVKVLPLSERN

SEQ ID NO: 42 - protein, 141 aa, Klebsiella pneumoniae
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 43 - protein, 471 aa, Klebsiella pneumoniae
MKNKWYKPKRHWKEIELWKDVPEEKWNDWLWQLTHTVRTLDDLKKVINLTEDEEEGVRISTKTIPLNITPYYASLMDPDNPRCPVRMQSVPLSEEMHKTKYDMEDPLHEDEDSPVPGLTHRYPDRVLFLVTNQCSVYCRHCTRRRFSGQIGMGVPKKQLDAAIAYIRETPEIRDCLLSGGDGLLINDQILEYILKELRSIPHLEVIRIGTRAPVVFPQRITDHLCEMLKKYHPVWLNTHFNTSIEMTEESVEACEKLVNAGVPVGNQAVVLAGINDSVPIMKKLMHDLVKIRVRPYYIYQCDLSEGIRHFRAPVSKGLEIIEGLRGHTSGYAVPTFVVHAPGGGGKIALQPNYVLSQSPDKVILRNFEGVITSYPEPENYIPNQADAYFESVFPETADKKEPIGLSALFADKEVSSTPENVDRIKRREAYIANPEHETLKDRREKRGQLKEKKFLAQQKKQKETECGGDSS

SEQ ID NO: 44 - protein, 292 aa, Bacillus cereus
MEHKTLSIGFIGIGVMGKSMVYHLMQDGHKVYVYNRTKAKTDSLVQDGAQWCDTPKELVKQVDIVMTMVGYPHDVEEVYFGIEGIIEHAKEGTIAIDFTTSTPTLAKRINEVAKSKNIYTLDAPVSGGDVGAKEAKLAIMVGGEKEIYDRCLPLLEKLGTNIQLQGPAGSGQHTKMCNQIAIASNMIGVCEAVAYAKKAGLNPDKVLESISTGAAGSWSLSNLAPRMLKGDFEPGFYVKHFMKDMKIALEEAEKLQLPVPGLSLAKELYEELIKDGEENSGTQVLYKKYIRG

SEQ ID NO: 45 - protein, 448 aa, Klebsiella pneumoniae
MNQPLNVAPPVSSELNLRAHWMPFSANRNFQKDPRIIVAAEGSWLTDDKGRKVYDSLSGLWTCGAGHSRKEIQEAVARQLGTLDYSPGFQYGHPLSFQLAEKIAGLLPGELNHVFFTGSGSECADTSIKMARAYWRLKGQPQKTKLIGRARGYHGVNVAGTSLGGIGGNRKMFGQLMDVDHLPHTLQPGMAFTRGMAQTGGVELANELLKLIELHDASNIAAVIVEPMSGSAGVLVPPVGYLQRLREICDQHNILLIFDEVITAFGRLGTYSGAEYFGVTPDLMNVAKQVTNGAVPMGAVIASSEIYDTFMNQALPEHAVEFSHGYTYSAHPVACAAGLAALDILARDNLVQQSAELAPHFEKGLHGLQGAKNVIDIRNCGLAGAIQIAPRDGDPTVRPFEAGMKLWQQGFYVRFGGDTLQFGPTFNARPEELDRLFDAVGEALNGIA

SEQ ID NO: 46 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 47 - protein, 551 aa, Escherichia coli
MNLWQQNYDPAGNIWLSSLIASLPILFFFFALIKLKLKGYVAASWTVAIALAVALLFYKMPVANALASVVYGFFYGLWPIAWIIIAAVFVYKISVKTGQFDIIRSSILSITPDQRLQMLIVGFCFGAFLEGAAGFGAPVAITAALLVGLGFKPLYAAGLCLIVNTAPVAFGAMGIPILVAGQVTGIDSFEIGQMVGRQLPFMTIIVLFWIMAIMDGWRGIKETWPAVVVAGGSFAIAQYLSSNFIGPELPDIISSLVSLLCLTLFLKRWQPVRVFRFGDLGASQVDMTLAHTGYTAGQVLRAWTPFLFLTATVTLWSIPPFKALFASGGALYEWVINIPVPYLDKLVARMPPVVSEATAYAAVFKFDWFSATGTAILFAALLSIVWLKMKPSDAISTFGSTLKELALPIYSIGMVLAFAFISNYSGLSSTLALALAHTGHAFTFFSPFLGWLGVFLTGSDTSSNALFAALQATAAQQIGVSDLLLVAANTTGGVTGKMISPQSIAIACAAVGLVGKESDLFRFTVKHSLIFTCIVGVITTLQAYVLTWMIP

SEQ ID NO: 48 - protein, 466 aa, Escherichia coli
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 49 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 50 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 51 - protein, 255 aa, Escherichia coli
MRHPLVMGNWKLNGSRHMVHELVSNLRKELAGVAGCAVAIAPPEMYIDMAKREAEGSHIMLGAQNVDLNLSGAFTGETSAAMLKDIGAQYIIIGHSERRTYHKESDELIAKKFAVLKEQGLTPVLCIGETEAENEAGKTEEVCARQIDAVLKTQGAAAFEGAVIAYEPVWAIGTGKSATPAQAQAVHKFIRDHIAKVDANIAEQVIIQYGGSVNASNAAELFAQPDIDGALVGGASLKADAFAVIVKAAEAAKQA

SEQ ID NO: 52 - protein, 85 aa, Escherichia coli
MFQQEVTITAPNGLHTRPAAQFVKEAKGFTSEITVTSNGKSASAKSLFKLQTLGLTQGTVVTISAEGEDEQKAVEHLVKLMAELE

SEQ ID NO: 53 - protein, 169 aa, Escherichia coli [sequence omitted]

SEQ ID NO: 54 - protein, 477 aa, Escherichia coli
MFKNAFANLQKVGKSLMLPVSVLPIAGILLGVGSANFSWLPAVVSHVMAEAGGSVFANMPLIFAIGVALGFTNNDGVSALAAVVAYGIMVKTMAVVAPLVLHLPAEEIASKHLADTGVLGGIISGAIAAYMFNRFYRIKLPEYLGFFAGKRFVPIISGLAAIFTGVVLSFIWPPIGSAIQTFSQWAAYQNPVVAFGIYGFIERCLVPFGLHHIWNVPFQMQIGEYTNAAGQVFHGDIPRYMAGDPTAGKLSGGFLFKMYGLPAAAIAIWHSAKPENRAKVGGIMISAALTSFLTGITEPIEFSFMFVAPILYIIHAILAGLAFPICILLGMRDGTSFSHGLIDFIVLSGNSSKLWLFPIVGIGYAIVYYTIFRVLIKALDLKTPGREDATEDAKATGTSEMAPALVAAFGGKENITNLDACITRLRVSVADVSKVDQAGLKKLGAAGVVVAGSGVQAIFGTKSDNLKTEMDEYIRNH

SEQ ID NO: 55 - protein, 575 aa, Escherichia coli
MISGILASPGIAFGKALLLKEDEIVIDRKKISADQVDQEVERFLSGRAKASAQLETIKTKAGETFGEEKEAIFEGHIMLLEDEELEQEIIALIKDKHMTADAAAHEVIEGQASALEELDDEYLKERAADVRDIGKRLLRNILGLKIIDLSAIQDEVILVAADLTPSETAQLNLKKVLGFITDAGGRTSHTSIMARSLELPAIVGTGSVTSQVKNDDYLILDAVNNQVYVNPTNEVIDKMRAVQEQVASEKAELAKLKDLPAITLDGHQVEVCANIGTVRDVEGAERNGAEGVGLYRTEFLFMDRDALPTEEEQFAAYKAVAEACGSQAVIVRTMDIGGDKELPYMNFPKEENPFLGWRAIRIAMDRREILRDQLRAILRASAFGKLRIMFPMIISVEEVRALRKEIEIYKQELRDEGKAFDESIEIGVMVETPAAATIARHLAKEVDFFSIGTNDLTQYTLAVDRGNDMISHLYQPMSPSVLNLIKQVIDASHAEGKWTGMCGELAGDERATLLLLGMGLDEFSMSAISIPRIKKIIRNTNFEDAKVLAEQALAQPTTDELMTLVNKFIEEKTIC

SEQ ID NO: 56 - protein, 510 aa, Escherichia coli
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALACVVGYYVVWNVSHALHTPLMSVTNAISGIIVVGALLQIGQGGWVSFLSFIAVLIASINIFGGFTVTQRMLKMFRKN

SEQ ID NO: 57 - protein, 339 aa, Escherichia coli
MNQRNASMTVIGAGSYGTALAITLARNGHEVVLWGHDPEHIATLERDRCNAAFLPDVPFPDTLHLESDLATALAASRNILVVVPSHVFGEVLRQIKPLMRPDARLVWATKGLEAETGRLLQDVAREALGDQIPLAVISGPTFAKELAAGLPTAISLASTDQTFADDLQQLLHCGKSFRVYSNPDFIGVQLGGAVKNVIAIGAGMSDGIGFGANARTALITRGLAEMSRLGAALGADPATFMGMAGLGDLVLTCTDNQSRNRRFGMMLGQGMDVQSAQEKIGQVVEGYRNTKEVRELAHRFGVEMPITEEIYQVLYCGKNAREAALTLLGRARKDERSSH

SEQ ID NO: 58 - protein, 510 aa, Escherichia coli; identical to SEQ ID NO: 56

SEQ ID NO: 59 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 60 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 61 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 62 - protein, 321 aa, Escherichia coli
MTKYALVGDVGGTNARLALCDIASGEISQAKTYSGLDYPSLEAVIRVYLEEHKVEVKDGCIAIACPITGDWVAMTNHTWAFSIAEMKKNLGFSHLEIINDFTAVSMAIPMLKKEHLIQFGGAEPVEGKPIAVYGAGTGLGVAHLVHVDKRWVSLPGEGGHVDFAPNSEEEAIILEILRAEIGHVSAERVLSGPGLVNLYRAIVKADNRLPENLKPKDITERALADSCTDCRRALSLFCVIMGRFGGNLALNLGTFGGVFIAGGIVPRFLEFFKASGFRAAFEDKGRFKEYVHDIPVYLIVHDNPGLLGSGAHLRQTLGHIL

SEQ ID NO: 63 - protein, 464 aa, Escherichia coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 64 - protein, 250 aa, Escherichia coli
MAVTKLVLVRHGESQWNKENRFTGWYDVDLSEKGVSEAKAAGKLLKEEGYSFDFAYTSVLKRAIHTLWNVLDELDQAWLPVEKSWKLNERHYGALQGLNKAETAEKYGDEQVKQWRRGFAVTPPELTKDDERYPGHDPRYAKLSEKELPLTESLALTIDRVIPYWNETILPRMKSGERVIIAAHGNSLRALVKYLDNMSEEEILELNIPTGVPLVYEFDENFKPLKRYYLGNADEIAAKAAAVANQGKAK

SEQ ID NO: 65 - protein, 432 aa, Escherichia coli
MSKIVKIIGREIIDSRGNPTVEAEVHLEGGFVGMAAAPSGASTGSREALELRDGDKSRFLGKGVTKAVAAVNGPIAQALIGKDAKDQAGIDKIMIDLDGTENKSKFGANAILAVSLANAKAAAAAKGMPLYEHIAELNGTPGKYSMPVPMMNIINGGEHADNNVDIQEFMIQPVGAKTVKEAIRMGSEVFHHLAKVLKAKGMNTAVGDEGGYAPNLGSNAEALAVIAEAVKAAGYELGKDITLAMDCAASEFYKDGKYVLAGEGNKAFTSEEFTHFLEELTKQYPIVSIEDGLDESDWDGFAYQTKVLGDKIQLVGDDLFVTNTKILKEGIEKGIANSILIKFNQIGSLTETLAAIKMAKDAGYTAVISHRSGETEDATIADLAVGTAAGQIKTGSMSRSDRVAKYNQLIRIEEALGEKAPYNGRKEIKGQA

SEQ ID NO: 66 - protein, 482 aa, Clostridium acetobutylicum
MFENISSNGVYKNLFDGKWVESKTNKTIETHSPYDGSLIGKVQALSKEEVDEIFKSSRTAQKKWGETPINERARIMRKAADILDDNAEYIAKILSNEIAKDLKSSLSEVKRTADFIRFTANEGTHMEGEAINSDNFPGSKKDKLSLVERVPLGIVLAISPFNYPVNLSGSKVAPALIAGNSVVLKPSTTGAISALHLAEIFNAAGLPAGVLNTVTGKGSEIGDYLITHEEVNFINFTGSSAVGKHISKIAGMIPMVLELGGKDAAIVLEDANLETTAKSIVSGAYGYSGQRCTAVKRVLVMDKVADELVELVTKKVKELKVGNPFDDVTITPLIDNKAADYVQTLIDDAIEKGATLIVGNKRKENLMYPTLFDNVTADMRIAWEEPFGPVLPIIRVKSMDEAIELANRSEYGLQSAVFTENMHDAFYIANKLDVGTVQVNNKPERGPDHFPFLGTKSSGMGTQGIRYSIEAMTRHKSIVLNL

(19) United States
(12) Patent Application Publication
(10) Pub. No.: US 2013/0224804 A1
(43) Pub. Date: Aug. 29, 2013

(54) EXPRESSION OF STEADY STATE METABOLIC PATHWAYS

(71) Applicant: Eric Knight, Cardiff, CA (US)

(72) Inventor: Eric Knight, Cardiff, CA (US)

(21) Appl. No.: 13/261,606

(22) PCT Filed: Sep. 1, 2011
(86) PCT No.: PCT/US11/50273

§ 371 Date: May 7, 2013

Related U.S. Application Data
(60) Provisional application No. 61/379,368, filed on Sep. 1, 2010.

(51) Int. Cl. C12P 19/02

(52) U.S. Cl. CPC C12P 19/02
USPC 435/105

(57) ABSTRACT

The present disclosure pertains to a method for increasing the production of a desired product having: identifying a steady state metabolic pathway for the synthesis of a desired product from a desired substrate; producing a polynucleotide encoding one or more polypeptide that participates in the steady state metabolic pathway for the synthesis of the desired product from the desired substrate; introducing the polynucleotide encoding a polypeptide into a host cell; transforming a host cell with an expression vector having an expressible polynucleotide encoding a polypeptide; and cultivating the host cell under a culture condition that induces the production of the desired product.

### CROSS-REFERENCE TO RELATED APPLICATION

This application claims the benefit of priority to U.S. Provisional Application No. 61/379,368, filed on Sep. 1, 2010, which is incorporated herein by reference in its entirety.

### Background

Concern about the environmental problems and limited nature of fossil resources, global demand for sustainable processes for the production of chemicals and materials from renewable biomass rather than from fossil fuel resources has been increasing. Microorganisms have been employed for the production of various chemicals and materials, however, their efficiencies and production rates are rather low when they are isolated from nature. Over the past few decades, the metabolic engineering of microorganisms has been successfully used to overcome this obstacle. Metabolic engineering is the application of engineering principles of design and analysis to the metabolic pathways in order to achieve a particular goal. This goal may be to increase process productivity, as in the case in production of antibiotics, biosynthetic precursors or polymers, or to extend metabolic capability by the addition of extrinsic activities for chemical production or degradation. Although metabolic engineering using the classical approach (i.e non-holistic approach)has contributed significantly to the enhanced production of various value-added and commodity chemicals and materials from renewable resources in the past two decades, recent advances in two emerging and highly synergistic fields, systems biology and synthetic biology, are allowing us to perform metabolic engineering more systematically and globally.

Systems biology aims at unraveling the underlying principles of biological systems through profiling the whole cellular characteristics using high-throughput technologies together with computational methods. Thus, systems biology continues to provide genome-wide information that facilitates metabolic engineering at various phases by predicting gene targets to be manipulated throughout the whole cellular network, which characterizes functional behavior of the biological system from a holistic perspective, and identifies novel biological entities that contribute to the enhanced production of chemicals and materials. In addition, the non-intuitive aspects of the biological system can be obtained from the theoretical counterpart of systems biology wherein rigorous modeling and simulation take place. Here, the theoretical systems biology allows mathematical description of the biological network that can be computationally simulated.

Synthetic biology aims at creating novel biologically functional parts, modules and systems by employing various molecular biology and synthetic DNA tools together with mathematical methodologies, and has been successfully applied in various metabolic engineering experiments. Several synthetic functions and modules have been developed to redirect metabolic pathways to produce novel metabolites; compute Boolean operations according to input signals; regulate metabolic fluxes in response to environmental changes; perform a specific biological behavior such as on/off switch and oscillation; and allow communication among cells. In addition, synthetic biology has greatly contributed to metabolic engineering by expanding the capacity of the production host, and thereby producing various chemicals and materials that are heterologous to the original host strain. Some example products that are produced by using synthetic biology include artemisinic acid, isopropanol, butanol, polylactic acid, glucaric acid, and various forms of alcohols, such as isobutanol, 1-butanol, 1-3 propanediol, 3-hydroxypropionic acid, and alkanes such as pentane and heptane.

Using the tools of system and synthetic biology, tremendous progress has been made in the area of metabolic engineering. These advances have allowed the conversion of renewable biomass sources such as glucose, cellubios, and hemicelluloses, into many chemicals such as organic acids, diols, alcohols, and hydrocarbons, which have thus far only been produced in large quantities from fossil resources. However, even though many of these chemicals are produced at very high yields, the production rates are inherently limited by the host organism's growth rate, since the organism must provide all cofactor balancing for the chemical production pathways within the organism. Every cofactor consumed by the chemical producing pathway creates a deficiency of the cofactor, and every cofactor produced by the chemical producing pathway creates an excess of the cofactor. In both cases, the reaction that created or consumed the cofactor will be significantly slowed by the cofactor imbalance, and will likely create a bottleneck in the chemical producing pathway.

### SUMMARY

The present disclosure pertains to a method for increasing the production of a desired product having: identifying a steady state metabolic pathway for the synthesis of a desired product from a desired substrate and expressing all polypeptides of the steady state metabolic pathway within a host cell.

One aspect of the disclosure pertains to a method for increasing the production of a desired product having: identifying a steady state metabolic pathway for the synthesis of a desired product from a desired substrate; producing a polynucleotide encoding one or more polypeptide that participates in the steady state metabolic pathway for the synthesis of the desired product from the desired substrate; introducing the polynucleotide encoding a polypeptide into a host cell; transforming a host cell with an expression vector having an expressible polynucleotide encoding a polypeptide; and cultivating the host cell under a culture condition that induces the production of the desired product.

One aspect of the method has collecting the desired product from the host cell. In another aspect of the disclosure the desired product is glucose. In another aspect of the disclosure the desired substrate is 3-Hydroxypropionic acid. In another aspect of the disclosure the host cell is Escherichia coli. In another aspect of the disclosure the host cell comprises a polynucleotide for T7 RNA polymerase.

One aspect of the disclosure pertains to a method for increasing the production of a desired product having: identifying a steady state metabolic pathway for the synthesis of a desired product from a desired substrate; producing a polynucleotide with nucleic acid sequences encoding all polypeptides that participate in the steady state metabolic pathway for the synthesis of the desired product from the desired substrate; introducing the polynucleotide encoding a polypeptide into a host cell; expressing the polynucleotides encoding all polypeptides of the steady state metabolic pathway; and cultivating the host cell under a culture condition that induces the production of the desired product.

In one aspect of the disclosure the one or more nucleic acid sequence encoding a polypeptide that participates in the steady state metabolic pathway is not incorporated into the polynucleotide.

With those and other objects, advantages and features on the present disclosure that may become hereinafter apparent, the nature of the present disclosure may be more clearly understood by reference to the following detailed description of the present disclosure, the appended claims, and the drawings attached hereto.

### BRIEF DESCRIPTION OF THE DRAWINGS

The accompanying drawings, which are incorporated herein and form part of the specification, illustrate various embodiments of the present disclosure and together with the description, further serve to explain the principles of the present disclosure and to enable a person skilled in the pertinent art to make and use the present disclosure. In the drawings, like reference numbers indicate identical or functionally similar elements. A more complete appreciation of the present disclosure and many of the attendant advantages thereof will be readily obtained as the same becomes better understood by reference to the following detailed description when considered in connection with the accompanying drawings, wherein:

FIG. 1 is a schematic drawing of a steady state metabolic pathway in E. Coli according to an exemplary embodiment.

FIG. 2 is a stoichiometric matrix according to an exemplary embodiment.

FIG. 3 is a table of net reaction rates according to an exemplary embodiment.

FIG. 4 is a schematic drawing of a vector according to an exemplary embodiment.

FIG. 5 is a schematic drawing of a steady state metabolic pathway in E. Coli according to an exemplary embodiment.

FIG. 6 is a stoichiometric matrix according to an exemplary embodiment.

FIG. 7 is a table of net reaction rates according to an exemplary embodiment.

FIG. 7 is a schematic drawing of a vector according to an exemplary embodiment.

FIG. 8 is a schematic drawing of a steady state metabolic pathway in E. Coli according to an exemplary embodiment.

FIG. 10 is a stoichiometric matrix according to an exemplary embodiment.

FIG. 11 is a table of net reaction rates according to an exemplary embodiment.

FIG. 12 is a schematic drawing of a vector according to an exemplary embodiment.

FIG. 13 is a schematic drawing of a steady state metabolic pathway in E. Coli according to an exemplary embodiment.

FIG. 14 is a stoichiometric matrix according to an exemplary embodiment.

FIG. 15 is a table of net reaction rates according to an exemplary embodiment.

FIG. 16 is a schematic drawing of a vector according to an exemplary embodiment.

FIG. 17 is a schematic drawing of a steady state metabolic pathway in E. Coli according to an exemplary embodiment.

FIG. 18 is a stoichiometric matrix according to an exemplary embodiment.

FIG. 19 is a table of net reaction rates according to an exemplary embodiment.

FIG. 20 is a schematic drawing of a vector according to an exemplary embodiment.

### DETAILED DESCRIPTION

In the following detailed description, reference is made to the accompanying drawings which form a part hereof and in which is shown by way of illustration specific embodiments in which the present disclosure may be practiced. These embodiments are described in sufficient detail to enable those skilled in the art to practice the present disclosure, and it is to be understood that other embodiments may be utilized and that structural or logical changes may be made without departing from the scope of the present disclosure. The following detailed description is, therefore, not to be taken in a limiting sense, and the scope of the present disclosure is defined by the appended claims.

The ability to investigate the metabolism of single cellular organisms at a genomic scale, in addition to recent advances in DNA construction, allows for novel methods for engineering microorganisms for the production of chemicals and biochemicals. The present disclosure combines recent advances in computation and experiment biology to express enzymes of steady state metabolic pathways in prokaryotic and eukaryotic cells for the production of chemicals and biochemicals.

Steady state metabolic pathways are self sustaining pathways that allow for the metabolic pathway to decouple from biomass production. This decoupling from biomass production allows a steady state metabolic pathway to perpetually synthesize a desired product. In other words, upon the presentation of a substrate, a steady state metabolic pathway can perpetuate the synthesis of a desired product independent of metabolites synthesized from metabolic pathways associated with biomass production.

It is possible to identify a steady state metabolic pathway without computational assistance, but given the vast number of reactions in current metabolic models, the computational procedure will identify not just straightforward but also non-intuitive strategies by simultaneously considering the entire metabolic network. An example of the size of current model is the in silico E. Coli model of Palsson and coworkers, which encompasses over 1200 reactions in the most recent version.

The optimization framework is developed to identify multiple gene combinations that maximize bioengineering objectives. This method can be applied for the maximization of the desired product based on a fixed amount of uptaken substrate. The method allows for the identification of enzymes to be expressed and their corresponding allowable envelopes of chemical production.

In one embodiment, the method allows for suggesting gene expression that could lead to chemical production in a host cell by ensuring that the drain towards metabolites/compounds must be accompanied, due to stoichiometry, by the production of a desired chemical. Specifically, the method identifies a steady state metabolic pathway that will increase production of a desired product, which can be realized by expressing the gene(s) associated with enzymes of the steady state metabolic pathway.

A plurality of steady state metabolic pathways can synthesize one desired product from a one desired substrate (e.g. production of Lactic acid, 3-Hydroxypropionic acid, 1,3-Propanediol, 1,2-Propanediol, Butanediol, Alkene Hydrocarbons, Alkane Hydrocarbons, Cycloalkane Hydrocarbons, from glucose, fructose, sucrose, galactose, cellobiose, maltose, hemicellulose, cellulose, starch, or the like), as described in the Examples herein. All steady state metabolic pathways used in the synthesis of one desired product from one desired substrate are anticipated. A plurality of steady state metabolic pathways can synthesize a plurality of desired products from a plurality of desired substrates (e.g. 3-Hydroxypropionic acid from glucose, 1,3-Propanediol acid from glucose, or the like). All steady state metabolic pathways used in the synthesis of a plurality of desired products from a plurality of desired substrates are anticipated.

The term “metabolic pathway” refers to any combination of catalytic activities, typically enzyme-mediated, that result in the chemical conversion of a substrate to a product. A metabolic pathway can be catabolic or anabolic. A metabolic pathway can be one that is normally found in a biological system, or can be a novel metabolic pathway not found in nature. A group of two or more enzymes are members of a common metabolic pathway if a substrate and/or product of each enzyme is a substrate or product for another member of the group, and the coordinated activities of the enzymes will, under the proper conditions, result in the conversion of a substrate to a product through an intermediate or series of intermediates. In a typical example, a substrate is converted into a first intermediate by a first member of the group, the first intermediate is converted into a second intermediate by a second member of the group, and the second intermediate is converted into the final product of the metabolic pathway by a third member of the group. The number of intermediates in a metabolic pathway varies with the pathway, e.g., some pathways have only a single intermediate. In some cases a metabolic pathway can branch, so that one or more intermediates can be converted into alternative products. Depending upon the metabolic pathway, the number of substrates, products and intermediates can vary from one to many.

The term “desired product” refers to compounds which are produced by a metabolic pathway. These compounds comprise organic acids, (e.g. 3-Hydroxypropionic acid, lactic acid, tartaric acid, itaconic acid and diaminopimelic acid), lipids, saturated and unsaturated fatty acids (e.g. arachidonic acid), diols (e.g. propanediol, 1,3-Propanediol, 1,2-Propanediol, and butanediol), alcohols (e.g. methanol, ethanol, isopropyl alcohol, butanol, pentanol) carbohydrates (e.g. hyaluronic acid and trehalose), aromatic compounds (e.g. benzene, aromatic amines, vanillin and indigo), vitamins and cofactors, alkene hydrocarbons (e.g. hexene, heptene, octene), alkane hydrocarbons (e.g. hexane, heptane, octane), cycloalkane hydrocarbons (e.g. cyclohexane, cycloheptane, cyclooctane), amino acidr (e.g. alanine, valine, tyrosine), or the like.

The term “desired substrate” refers to compounds in which an enzyme acts and are used in the first step of a metabolic pathway. These compounds comprise glucose, fructose, sucrose, galactose, cellobiose, maltose, hemicellulose, cellulose, starch, or the like.

The present disclosure provides for methods of increasing the production of a desired product synthesized from a metabolic pathway. In one embodiment, the desired product is produced by identifying a steady state metabolic pathway that produces the desired product, synthesizing a polynucleotide that encodes for at least one polypeptide found in the steady state metabolic pathway, and expressing the polynucleotide.

In order to identify a steady state metabolic pathway, a metabolic network with m compounds and n metabolic reactions is considered. One can define the topology of the resulting hypergraph using a generalized incidence matrix, S ∈ Z^{m,n}. Each row in this stoichiometric matrix represents a particular compound, e.g. glucose, while each column represents a chemical reaction. With respect to the forward direction of a reaction, for all i=1 . . . m and j=1 . . . n, S_{i,j}<0 if compound i is a substrate in a reaction, meaning that it is consumed by the reaction j, S_{i,j}>0 if compound i is a product, meaning that it is produced by a reaction, and S_{i,j}=0 otherwise. Typically stoichiometric coefficients are integers reflecting the number of copies of a compound consumed or produced in a reaction. Each column of S corresponds to a mass conserving chemical reaction, except for certain exchange reactions that do not conserve mass. Exchange reactions are a modeling abstraction used to represent the exchange of mass across the boundary of a system.

The inner product of the stoichiometric matrix S and a vector of net reaction rates v in Rⁿ, gives the change in concentration over time of each metabolite, S·v=dx/dt, where x represents concentration and t represents time. Assuming that a biochemical reaction network operates at a steady state, we have S·v=dx/dt=0, which is defined here as a steady state metabolic pathway. The set of all reaction rates that satisfy steady state (i.e. all steady state metabolic pathways) is contained in the polyhedral cone defined by S·v=0. There is a bijective correspondence between each metabolic pathway and each extreme ray of the aforementioned polyhedral cone.

Various methods can be employed to compute a steady state metabolic pathway that corresponds to the maximization of a particular bioengineering objective. Such a bioengineering objective could be, for example, without limitation, the maximization of an exchange reaction rate(s), such as maximum growth rate, maximum synthesis rate of a desired product or combination of products, or the like. Various optimization or extreme ray enumeration algorithms can be used to identify a steady state metabolic pathway maximizing a bioengineering objective. Flux balance analysis (FBA) is one such method for identifying a steady state metabolic pathway maximizing a bioengineering objective.

### Polynucleotide Compositions

The scope of the present disclosure with respect to polynucleotide compositions can include, for example, without limitation, polynucleotides having a sequence set forth in at least one of SEQ ID NOS: 1-38; polynucleotides obtained from the biological materials described herein or other biological sources; genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present disclosure will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicted.

Nucleic acid compositions of the present disclosure of particular interest comprise a sequence set forth in at least one of SEQ ID NOS:1-38 or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject novel nucleic acid compositions include full length cDNAs or mRNAs that encompass an identifying sequence of contiguous nucleotides from at least one of SEQ ID NOS: 1-38.

The polynucleotides of the present disclosure also include polynucleotides having sequence similarity or sequence identity, for example, variants, (e.g., degenerate variants, allelic variants, etc.) genetically altered versions of the gene, homologous genes, or related genes of at least one SEQ ID NOS:1-38. Allelic variants can exhibit at most about 25-30% base pair (bp) mismatches relative to the selected polynucleotide probe. Allelic variants contain 15-25% by mismatches, and can contain as little as even 5-15%, or 2-5%, or 1-2% by mismatches, as well as a single by mismatch. Variants of the present disclosure have a sequence identity greater than at least about 65%, preferably at least about 75%, more preferably at least about 85%, and can be greater than at least about 90. Homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity, e.g., at least 75% sequence identity, usually at least 90%, more usually at least 95% between nucleotide sequences.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The polynucleotides incorporated into the DNA construct can be directly linked to one another, or the polynucleotides can be separated by nucleotide linker sequences. Separation of the component enzymatic activities can be accomplished, for example, through the use of peptide linkers that are sensitive to proteolytic cleavage or hydrolysis, or by incorporation of intein or intron sequences into the linker sequences.

The nucleic acid compositions of the present disclosure can encode all or a part of the subject polypeptides. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments of the present disclosure comprise at least about 10, about 15, about 20, about 35, about 50, about 100, about 150 to about 200, about 250 to about 300, or about 350 contiguous nt selected from the polynucleotide sequences as shown in SEQ ID NOS:1-38. Typically, fragments will be of at least 15 nt, usually at least 18 nt or 25 nt, and up to at least about 50 contiguous nt in length or more. In a preferred embodiment, the polynucleotide molecules comprise a contiguous sequence of at least 12 nt selected from the group consisting of the polynucleotides shown in SEQ ID NOS:1-38

The polynucleotides of the subject present disclosure are isolated and obtained in substantial purity, generally as other than an intact chromosome. Usually, the polynucleotides, either as DNA or RNA, will be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

The polynucleotides of the present disclosure can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides of the present disclosure can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The subject nucleic acid compositions can be used to, for example, to produce polypeptides, as enzymes used in a metabolic pathway to generate a desired compound.

Full-Length cDNA, Gene, and Promoter Region

Full-length cDNA molecules having a sequence of at least one of SEQ ID NOS:1-38 are obtained as follows. Libraries of cDNA are made from selected tissues, such as normal or tumor tissue, or from tissues of a mammal treated with, for example, a pharmaceutical agent. Preferably, the tissue is the same as the tissue from which the polynucleotides of the present disclosure were isolated, as both the polynucleotides described herein and the cDNA represent expressed genes. Most preferably, the cDNA library is made from the biological material described herein. The choice of cell type for library construction can be made after the identity of the protein encoded by the gene corresponding to the polynucleotide of the present disclosure is known. This will indicate which tissue and cell types are likely to express the related gene, and thus represent a suitable source for the mRNA for generating the cDNA. Where the provided polynucleotides are isolated from cDNA libraries, the libraries are prepared from mRNA of human colon cells.

The cDNA can be prepared by using primers based on sequence from at least one SEQ ID NOS:1-38.

Members of the library that are larger than the provided polynucleotides, and preferably that encompass the complete coding sequence of the native message, are obtained. In order to confirm that the entire cDNA has been obtained, RNA protection experiments are performed as follows. Hybridization of a full-length cDNA to an mRNA will protect the RNA from RNase degradation. If the cDNA is not full length, then the portions of the mRNA that are not hybridized will be subject to RNase degradation. This is assayed, as is known in the art, by changes in electrophoretic mobility on polyacrylamide gels, or by detection of released monoribonucleotides. In order to obtain additional sequences 5′ to the end of a partial cDNA, 5′ RACE can be performed.

Genomic DNA is isolated using the provided polynucleotides in a manner similar to the isolation of full-length cDNAs. Briefly, the provided polynucleotides, or portions thereof, are used as probes to libraries of genomic DNA. Preferably, the library is obtained from the cell type that was used to generate the polynucleotides of the present disclosure, but this is not essential. Most preferably, the genomic DNA is obtained from the biological material described herein. Such libraries can be in vectors suitable for carrying large segments of a genome, such as P1 or YAC. In addition, genomic sequences can be isolated from human BAC (bacterial artificial chromosome) libraries. In order to obtain additional 5′ or 3′ sequences, chromosome walking is performed, such that adjacent and overlapping fragments of genomic DNA are isolated. These are mapped and pieced together, as is known in the art, using restriction digestion enzymes and DNA ligase.

Using the polynucleotide sequences of the present disclosure, corresponding full-length genes can be isolated using both classical and PCR methods to construct and probe cDNA libraries. Using either method, Northern blots, preferably, are performed on a number of cell types to determine which cell lines express the gene of interest at the highest level. Classical methods of constructing cDNA libraries are taught. With these methods, cDNA can be produced from mRNA and inserted into viral or expression vectors. Typically, libraries of mRNA comprising poly(A) tails can be produced with poly(T) primers. Similarly, cDNA libraries can be produced using the instant sequences as primers.

PCR methods are used to amplify the members of a cDNA library that comprise the desired insert. In this case, the desired insert will contain sequence from the full length cDNA that corresponds to the instant polynucleotides. Such PCR methods include gene trapping and RACE methods.

Another PCR-based method generates full-length cDNA library with anchored ends without needing specific knowledge of the cDNA sequence. The method uses lock-docking primers (I-VI), where one primer, poly TV (I-III) locks over the polyA tail of eukaryotic mRNA producing first strand synthesis and a second primer, polyGH (IV-VI) locks onto the polyC tail added by terminal deoxynucleotidyl transferase (TdT).

Once the full-length cDNA or gene is obtained, DNA encoding variants can be prepared by site-directed mutagenesis. The choice of codon or nucleotide to be replaced can be based on disclosure herein on optional changes in amino acids to achieve altered protein structure and/or function.

As an alternative method to obtaining DNA or RNA from a biological material, nucleic acid comprising nucleotides having the sequence of one or more polynucleotides of the present disclosure can be synthesized. Thus, the present disclosure encompasses nucleic acid molecules ranging in length from 15 nt (corresponding to at least 15 contiguous nt of at least one of SEQ ID NOS:1-38) up to a maximum length suitable for one or more biological manipulations, including replication and expression, of the nucleic acid molecule. The present disclosure can include, for example, without limitation, (a) a nucleic acid having the size of a full gene, and comprising at least one of SEQ ID NOS:1-38; (b) an expression vector comprising (a); (c) a plasmid comprising (a); and (d) a recombinant viral particle comprising (a). Once provided with the polynucleotides disclosed herein, construction or preparation of (a)-(d) are well within the skill in the art.

The sequence of a nucleic acid comprising at least 15 contiguous nt of at least one of SEQ ID NOS:1-38, preferably the entire sequence of at least one of SEQ ID NOS:1-38, is not limited and can be any sequence of A, T, G, and/or C (for DNA) and A, U, G, and/or C (for RNA) or modified bases thereof, including inosine and pseudouridine. The choice of sequence will depend on the desired function and can be dictated by coding regions desired, the intron-like regions desired, and the regulatory regions desired. Where the entire sequence of at least one of SEQ ID NOS:1-38 is within the nucleic acid, the nucleic acid obtained is referred to herein as a polynucleotide comprising the sequence of at least one of SEQ ID NOS:1-38.

### Polypeptides and Variants Thereof

The polypeptides of the present disclosure include those encoded by the disclosed polynucleotides, as well as nucleic acids that, by virtue of the degeneracy of the genetic code, are not identical in sequence to the disclosed polynucleotides. Thus, the present disclosure includes within its scope a polypeptide encoded by a polynucleotide having the sequence of at least one of SEQ ID NOS:1-38 or a variant thereof. A polypeptide of present disclosure includes, for example, the protein whose sequence is provided in at least one SEQ ID NO:39-66, or any variant thereof, while still encoding a protein that maintains like activities and physiological functions, or a functional fragment thereof.

In general, the term “polypeptide” as used herein refers to both the full length polypeptide encoded by the recited polynucleotide, the polypeptide encoded by the gene represented by the recited polynucleotide, as well as portions or fragments thereof. “Polypeptides” also includes variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, usually at least about 90%, and more usually at least about 98% sequence identity with a differentially expressed polypeptide of the present disclosure. The variant polypeptides can be naturally or non-naturally glycosylated, i.e., the polypeptide has a glycosylation pattern that differs from the glycosylation pattern found in the corresponding naturally occurring protein.

The present disclosure also encompasses homologs of the disclosed polypeptides (or fragments thereof) where the homologs are isolated from other species, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular differentially expressed protein.

The polypeptides of the present disclosure can be provided in a non-naturally occurring environment, e.g. separated from their naturally occurring environment. In certain embodiments, the subject protein is present in a composition that is enriched for the protein as compared to a control. As such, purified polypeptide is provided, where by purified is meant that the protein is present in a composition that is substantially free of non-differentially expressed polypeptides, where by substantially free is meant that less than 90%, usually less than 60% and more usually less than 50% of the composition is made up of non-differentially expressed polypeptides.

Also within the scope of the present disclosure are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid the thermostability of the variant polypeptide, desired glycosylation sites, desired disulfide bridges, desired metal binding sites, and desired substitutions with in proline loops. Cysteine-depleted muteins can be produced as disclosed in U.S. Pat. No. 4,959,314.

Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of at least one SEQ ID NOS:1-38, or a homolog thereof. The protein variants described herein are encoded by polynucleotides that are within the scope of the present disclosure. The genetic code can be used to select the appropriate codons to construct the corresponding variants.

Recombinant Expression Vectors and Host Cells

Another aspect of the present disclosure pertains to vectors, preferably expression vectors, containing a nucleic acid encoding a protein, or derivatives, fragments, analogs or homologs thereof. As used herein, the term “vector” refers to a nucleic acid molecule capable of transporting another nucleic acid to which it has been linked One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The recombinant expression vectors of the present disclosure comprise a nucleic acid of the present disclosure in a form suitable for expression of the nucleic acid in a host cell, thereby meaning that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the present disclosure can be introduced into host cells to thereby produce proteins or peptides, including fusion proteins or peptides, encoded by nucleic acids as described herein.

The recombinant expression vectors of the present disclosure can be designed for expression of proteins in prokaryotic or eukaryotic cells. For example, proteins can be expressed in bacterial cells such as Escherichia coli, insect cells (using baculovirus expression vectors) yeast cells or mammalian cells. In one embodiment, the recombinant expression vector can be transcribed and translated in vitro, for example, using T7 promoter regulatory sequences and T7 polymerase.

In another embodiment, the expression vector is a yeast expression vector. In one embodiment, polynucleotides can be expressed in insect cells using baculovirus expression vectors. Baculovirus vectors available for expression of proteins in cultured insect cells (e.g., SF9 cells) include the pAc series and the pVL series.

In yet another embodiment, a nucleic acid of the present disclosure is expressed in mammalian cells using a mammalian expression vector. Examples of mammalian expression vectors include pCDM8 and pMT2PC.

The present disclosure further provides a recombinant expression vector comprising a DNA molecule of the present disclosure cloned into the expression vector in an antisense orientation. That is, the DNA molecule is operatively-linked to a regulatory sequence in a manner that allows for expression (by transcription of the DNA molecule) of an RNA molecule that is antisense to mRNA associated with the metabolic pathway enzymes. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the present disclosure pertains to host cells into which a recombinant expression vector of the present disclosure has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, protein can be expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the metabolic pathway enzymes or can be introduced on a separate vector. Cells stably transfected with the introduced nucleic acid can be identified by drug selection (e.g., cells that have incorporated the selectable marker gene will survive, while the other cells die).

A host cell of the present disclosure, such as a prokaryotic or eukaryotic host cell in culture, can be used to produce (i.e., express) protein. Accordingly, the present disclosure further provides methods for producing protein using the host cells of the present disclosure. In one embodiment, the method comprises culturing the host cell of present disclosure (into which a recombinant expression vector encoding protein has been introduced) in a suitable medium such that protein is produced. In another embodiment, the method further comprises isolating protein from the medium or the host cell.

Expression of Polypeptide Encoded by Full-Length cDNA or Full-Length Gene

The provided polynucleotides (e.g., a polynucleotide having a sequence of at least one SEQ ID NOS:1-38), the corresponding cDNA, or the full-length gene is used to express a partial or complete gene product. Constructs of polynucleotides having sequences of at least one SEQ ID NOS:1-38 can also be generated synthetically. Alternatively, single-step assembly of a gene and entire plasmid from large numbers of oligodeoxyribonucleotides is derived from DNA shuffling, and does not rely on DNA ligase, but instead relies on DNA polymerase to build increasingly longer DNA fragments during the assembly process.

Appropriate polynucleotide constructs are purified using standard recombinant DNA techniques. The gene product encoded by a polynucleotide of the present disclosure is expressed in any expression system, including, for example, bacterial, yeast, insect, amphibian and mammalian systems.

The polynucleotides set forth in SEQ ID NOS:1-38 or their corresponding full-length polynucleotides are linked to regulatory sequences as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used.

When any of the above host cells, or other appropriate host cells or organisms, are used to replicate and/or express the polynucleotides or nucleic acids of the present disclosure, the resulting replicated nucleic acid, RNA, expressed protein or polypeptide, is within the scope of the present disclosure as a product of the host cell or organism. The host cells are cultivated in a suitable medium and he product is recovered by any appropriate means known in the art.

In some embodiments, the method has secretion routes for transporting the desired product or other metabolites across a cell wall or cell membrane, for example, a transport reaction, hydrogen symporter, diffusion, or the like. In one embodiment, the secretion routes allow for the presence of the steady state metabolic pathway. In one embodiment, separate optimizations can be run for all potential transport mechanisms to identify unknown transport mechanisms.

The desired product is determined by traditional analytical techniques for example, without limitation, mass spectrometry, thin layer chromatography (TLC), high pressure liquid chromatography (HPLC), capillary electrophoresis (CE), and NMR spectroscopy.

Lactic Acid Synthesis using a Steady State Metabolic Pathway

The synthesis of Lactic acid from glucose in a steady state metabolic pathway in Escherichia coli is performed. In one embodiment, a steady state metabolic pathway in Escherichia coli for the synthesis of lactic acid from glucose is identified. A constraint based model of Escherichia coli metabolism is used to determine a steady state metabolic pathway for the synthesis of lactic acid from glucose in Escherichia coli using Escherichia coli model iAF1260 (Feist A M, et al, Mol Syst Biol. 2007; 3:121.Feist). NADP-dependent glyceraldehyde-3-phosphate dehydrogenase from Clostridium acetobutylicum (GAPN(SEQ ID NO 69)) added to the model to allow for a more simplistic pathway. FBA is used to identify a steady state metabolic pathway by maximizing for lactic acid, using glucose as a substrate. The glucose exchange reaction is set in the FBA to allow the uptake of 1 mole of glucose/hour (M/h). The exchange reactions for 3-Lactic acid, oxygen, water, and carbon dioxide, are set in the FBA to allow the uptake and secretion of these metabolites to be unbounded.

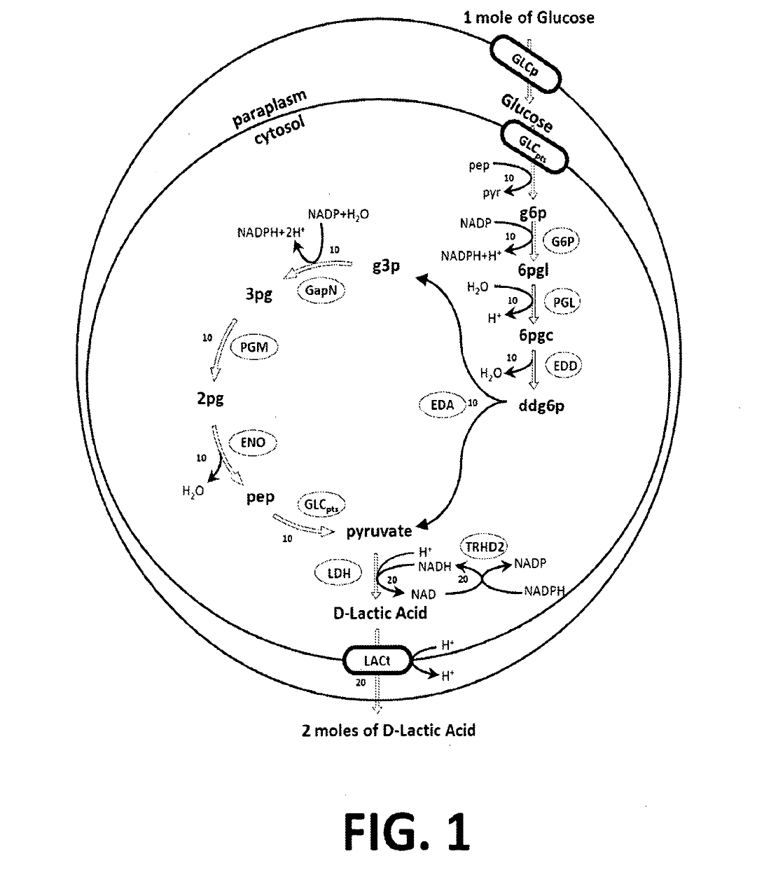

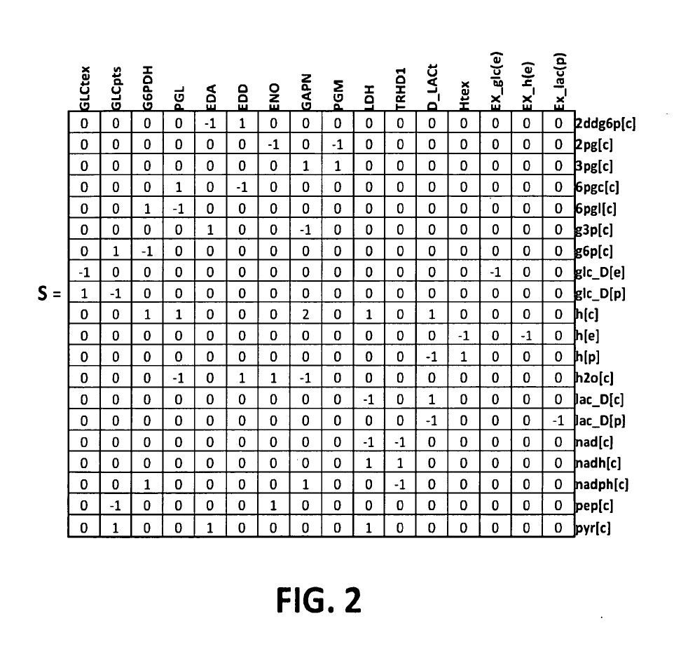

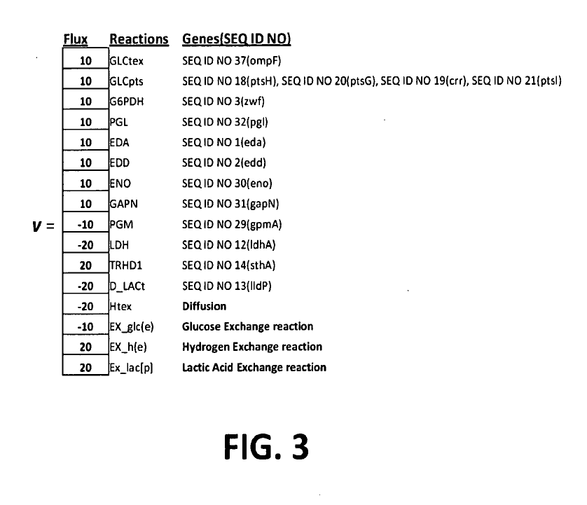

In Escherichia coli, there are many steady state metabolic pathways for the synthesis of lactic acid, using glucose as a desired substrate. FIG. 1 shows one steady state metabolic pathway for the synthesis of lactic acid, using glucose as a desired substrate, defined as LACBAC, having the reactions 2-keto-3-deoxygluconate 6-phosphate aldolase from Escherichia coli (EDA(SEQ ID NO 39)), phosphogluconate dehydratase from Escherichia coli (EDD(SEQ ID NO 40)), glucose 6-phosphate-1-dehydrogenase from Escherichia coli (G6P(SEQ ID NO 41)), lactate dehydrogenase from Escherichia coli (LDHA(SEQ ID NO 50)), lactate/proton symporter from Escherichia coli (LLDP(SEQ ID NO 51)), glucose-specific PTS permease from Escherichia coli (GLCpts(PTSH(SEQ ID NO 56), CRR(SEQ ID NO 57), PTSG(SEQ ID NO 58), PTSI (SEQ ID NO 59))), 2,3-bisphosphoglycerate-dependent phosphoglycerate mutase from Escherichia coli (GPMA(SEQ ID NO 67)), enolase from Escherichia coli (ENO(SEQ ID NO 68)), NADP-dependent glyceraldehyde-3-phosphate dehydrogenase from Clostridium acetobutylicum (GAPN(SEQ ID NO 69)), 6-phosphogluconolactonase from Escherichia coli (PGL(SEQ ID NO 70)), and outer membrane porin F from Escherichia coli (OMPF(SEQ ID NO 75)). For the synthesis of lactic acid from glucose in Escherichia coli, stoichiometric matrix (S) and flux vector (v) and of the steady state metabolic pathway are shown in FIGS. 2 and 3, respectively, demonstrating that S·v=0 and LACBAC is a steady state metabolic pathway.

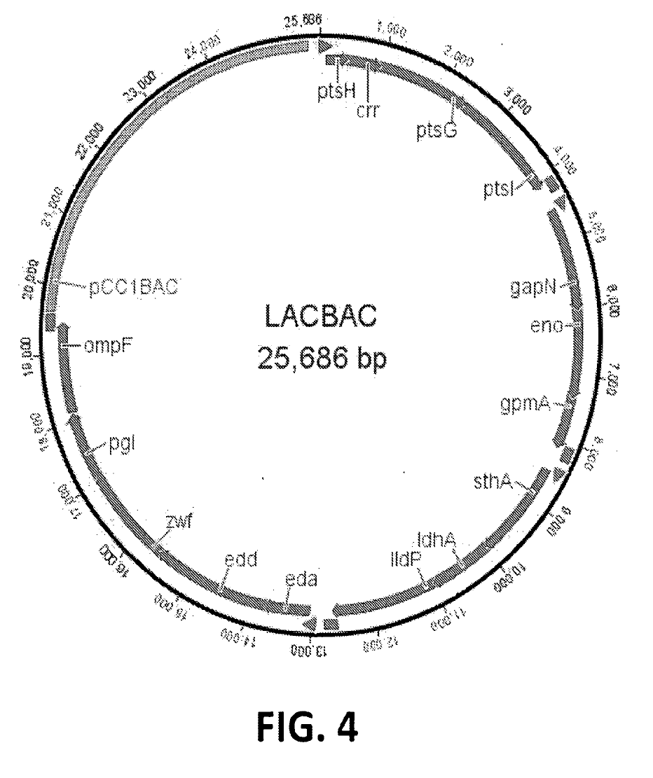

In one embodiment, the metabolic pathway DNA construct for the LACBAC design, shown in FIG. 4, is created that has a sequence set forth in the following SEQ ID NOS: SEQ ID NO 37 (ompF), SEQ ID NO 18 (ptsH), SEQ ID NO 20 (ptsG), SEQ ID NO 19 (crr), SEQ ID NO 21 (ptsl), SEQ ID NO 3 (zwf), SEQ ID NO 32 (pgl), SEQ ID NO 1 (eda), SEQ ID NO 2 (edd), SEQ ID NO 30 (eno), SEQ ID NO 31 (gapN), SEQ ID NO 29 (gpmA), SEQ ID NO 12 (ldhA), SEQ ID NO 14 (TRHD1), and SEQ ID NO 13 (11dP).

Once a steady state metabolic pathway for the synthesis of lactic acid from glucose has been identified, the enzymes of the steady state metabolic pathway are expressed in a host cell. A metabolic pathway DNA construct is created with each polynucleotide that encodes an enzyme of the 3HP1BAC steady state metabolic pathway. All enzymes are synthesized from a T7 RNA polymerase, thus allowing induction using Isopropyl β-D-1-thiogalactopyranoside (IPTG). A 4 chew-back, anneal and repair (CBAR) reaction buffer (20% PEG-8000, 600 mM Tris-HCl pH 7.5, 40 mM MgCl2, 40 mMDTT, 800 mM each of the four dNTPs and 4 mM NAD) is used for one-step thermocycled DNA assembly. DNA constructs are assembled in 40 ml reactions consisting of 10 ml 4 CBAR buffer, 0.35 ml of 4 U ml/l ExoIII (NEB), 4 ml of 40 U/ml Taq DNA ligase and 0.25 ml of 5 U/ml Ab-Taq polymerase. ExoIII is diluted 1:25 from 100 U ml/l in its stored buffer (50% glycerol, 5 mM KPO4, 200 mM KCl, 5 mM 2-mercaptoethanol, 0.05 mM EDTA and 200 mg ml/l BSA, pH 6.5). DNA construct reactions are prepared in 0.2 ml PCR tubes and cycled using the following conditions: 37 C for 5 or 15 min, 75 C for 20 min, −0.1 C/second to 60 C, then held at 60 C for 1 h. In general, a chew-back time of 5 min was used for overlaps less than 80 by and 15 min for overlaps greater than 80 bp. The base pairs used in the DNA construct assembly are generated from restriction digestion of DNA, synthetically synthesized DNA, and PCR products derived from plasmids and genomic DNA. All DNA base pairs have overlapping regions, which enable the assembly of the multiple DNA constructs into a single DNA construct. The DNA base pairs are integrated together in a linearized pcc1BAC, and thus the final assembly is a BAC able to replicate in a host cell.

The DNA construct is then introduced into an Escherichia coli host cell harboring the T7 RNA polymerase, such as BL21 and BL21 Lys. Isopropyl β-D-1-thiogalactopyranoside (IPTG) is used to induce the production of T7 RNA polymerase, which in turn, induces the expression of all genes on the metabolic pathway DNA construct under T7 RNA polymerase control. The metabolic pathway DNA construct can then be expressed to produce the steady state metabolic pathway enzymes encoded by a polynucleotide.

The desired lactic acid product is determined by traditional analytical techniques for example as described herein.

3-Hydroxypropionic Acid Synthesis using a Steady State Metabolic Pathway with Diffusion Transport of 3-Hydroxypropionic Acid: 3HP1BAC Design

The synthesis of 3-Hydroxypropionic acid from glucose in a steady state metabolic pathway in Escherichia coli is performed. In one embodiment, a steady state metabolic pathway in Escherichia coli for the synthesis of 3-Hydroxypropionic acid from glucose is identified. A constraint based model of Escherichia coli metabolism is used to determine a steady state metabolic pathway for the synthesis of 3-Hydroxypropionic acid from glucose in Escherichia coli using Escherichia coli model iAF1260 (Feist A M, et al, Mol Syst Biol. 2007; 3:121.Feist). 3-Hydroxypropionic acid is not naturally produced in Escherichia coli and thus the following reactions identified using the KEG database are added to the Escherichia coli model: glycerol dehydratase from Klebsiella pneumonia (DHAB containing the subunits (DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)), NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)). The pyruvate kinase II (PYKA(SEQ ID NO 76)) in the iAF1260 model is made reversible. In addition, a transport reaction is added to the iAF1260 model. For this example, it is assumed that 3-Hydroxypropionic acid is transported out of the Escherichia coli cell via diffusion, and the diffusion reaction (3HP1t) is added to the iAF1260 model. FBA is used to identify a steady state metabolic pathway by maximizing for 3-Hydroxypropionic acid, using glucose as a desired substrate. The glucose exchange reaction is set in FBA to allow the uptake of 1 mole of glucose/hour (M/h). The exchange reactions for 3-Hydroxypropionic acid, oxygen, water, and carbon dioxide, are set in FBA to allow the uptake and secretion of these metabolites to be unbounded.

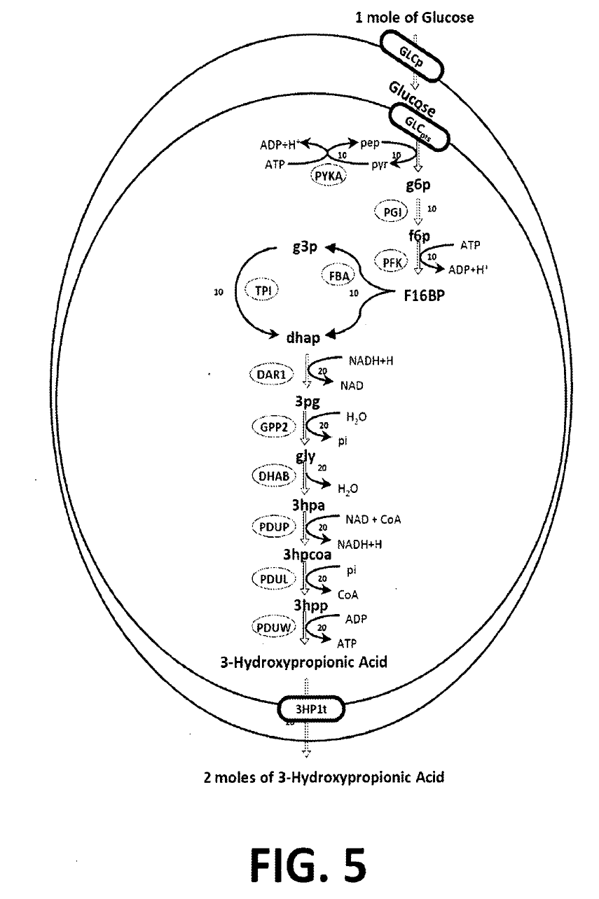

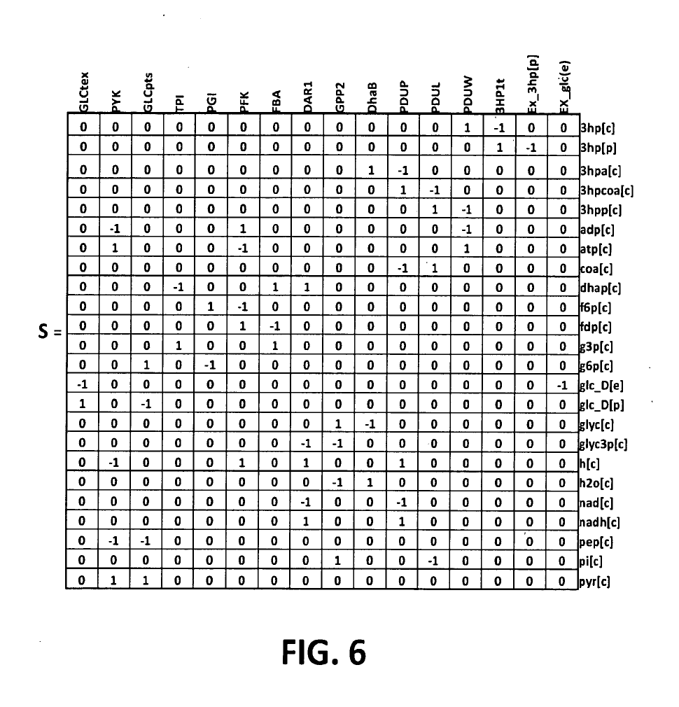

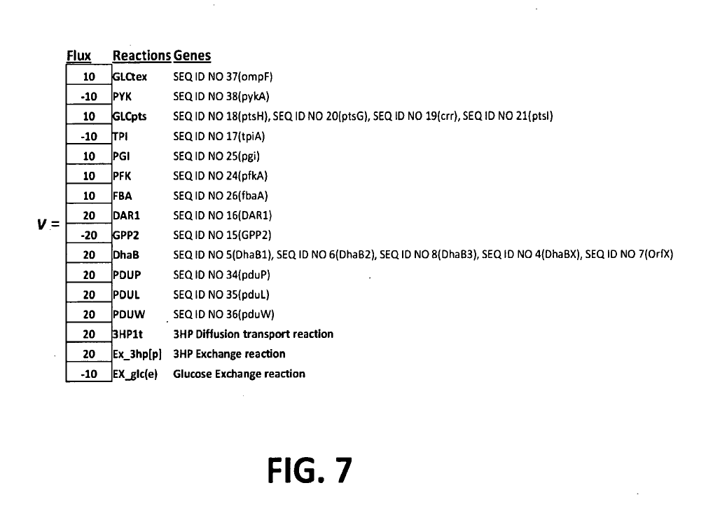

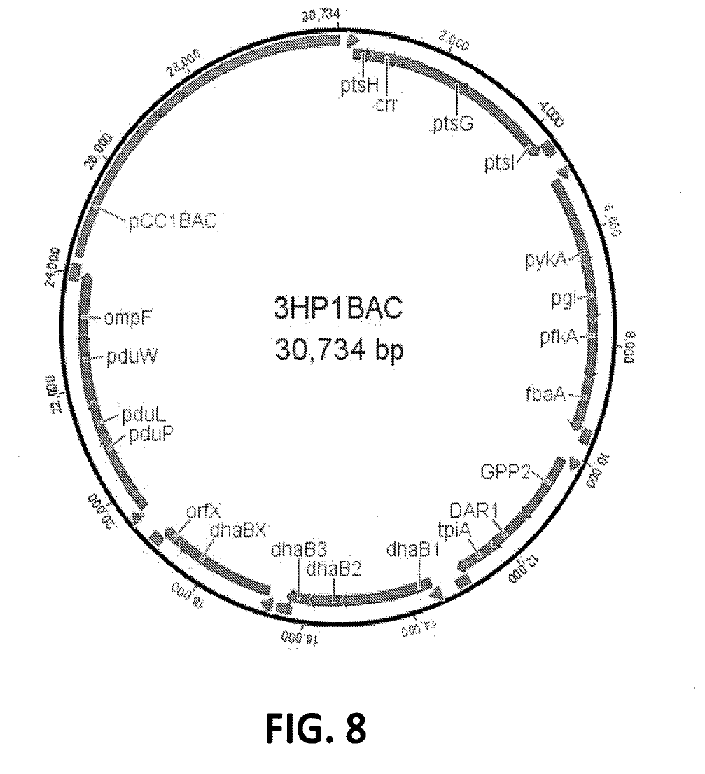

With added reactions to the iAF1260 model, there are many steady state metabolic pathways for the synthesis of 3-Hydroxypropionic acid, using glucose as a desired substrate. FIG. 5 shows one steady state metabolic pathway for the synthesis of 3-Hydroxypropionic acid, using glucose as a desired substrate, defined as 3HP1BAC, having the reactions glycerol dehydratase from Klebsiella pneumonia (DHAB containing the subunits (DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)), NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)), triose phosphate isomerase from Escherichia coli (TPIA(SEQ ID NO 55)), glucose-specific PTS permease from Escherichia coli (PTSH(SEQ ID NO 56), CRR(SEQ ID NO 57), PTSG(SEQ ID NO 58), PTSI (SEQ ID NO 59)), 6-phosphofructokinase I from Escherichia coli (PFKA(SEQ ID NO 62)), phosphoglucose isomerase from Escherichia coli (PGI(SEQ ID NO 63)), fructose bisphosphate aldolase class II from Escherichia coli (FBAA(SEQ ID NO 64)), outer membrane porin F from Escherichia coli (OMPF(SEQ ID NO 75)), pyruvate kinase II from Escherichia coli (PYKA(SEQ ID NO 76)), and the 3HP1t transport reaction. For the synthesis of 3-Hydroxypropionic acid from glucose in Escherichia coli, stoichiometric matrix (S) and flux vector (v) and of the steady state metabolic pathway are shown in FIGS. 6 and 7, respectively demonstrating that S·v=0 and 3HP1BAC metabolic pathway is a steady state metabolic pathway.

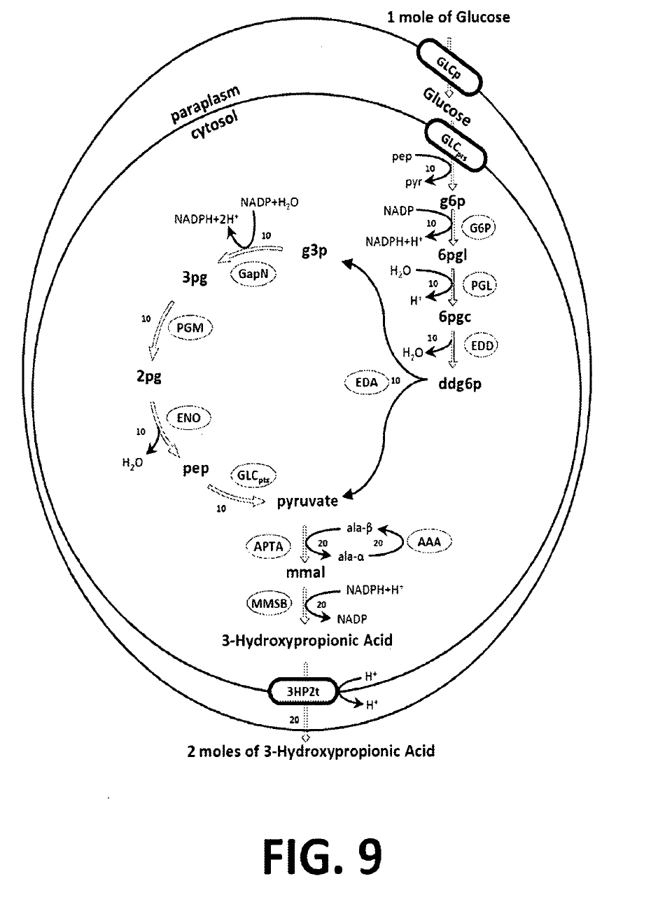

In one embodiment, the metabolic pathway DNA construct for the 3HP1BAC design, shown in FIG. 8, is created that has a sequence set forth in the following SEQ ID NOS: SEQ ID NO 37 (ompF), SEQ ID NO 38 (pykA), SEQ ID NO 18 (ptsH), SEQ ID NO 20 (ptsG), SEQ ID NO 19 (crr), SEQ ID NO 21 (ptsl), SEQ ID NO 17 (tpiA), SEQ ID NO 25 (pgi), SEQ ID NO 24 (pfkA), SEQ ID NO 26 (fbaA), SEQ ID NO 16 (DAR1), SEQ ID NO 15 (GPP2), SEQ ID NO 5 (DhaB1), SEQ ID NO 6 (DhaB2), SEQ ID NO 8 (DhaB3), SEQ ID NO 4 (DhaBX), SEQ ID NO 7 (OrfX), SEQ ID NO 34 (pduP), SEQ ID NO 35 (pduL), and SEQ ID NO 36 (pduW).

Once a steady state metabolic pathway for the synthesis of 3-Hydroxypropionic acid from glucose has been identified, the enzymes of the steady state metabolic pathway are expressed in a host cell. A metabolic pathway DNA construct is created with each polynucleotide that encodes an enzyme of the 3HP1BAC steady state metabolic pathway. All enzymes are synthesized from a T7 RNA polymerase, thus allowing induction using Isopropyl β-D-1-thiogalactopyranoside (IPTG). A 4 chew-back, anneal and repair (CBAR) reaction buffer (20% PEG-8000, 600 mM Tris-HCl pH 7.5, 40 mM MgCl2, 40 mMDTT, 800 mM each of the four dNTPs and 4 mM NAD) is used for one-step thermocycled DNA assembly. DNA constructs are assembled in 40 ml reactions consisting of 10 ml 4 CBAR buffer, 0.35 ml of 4 U ml/l ExoIII (NEB), 4 ml of 40 U/ml Taq DNA ligase and 0.25 ml of 5 U/ml Ab-Taq polymerase. ExoIII is diluted 1:25 from 100 U ml/l in its stored buffer (50% glycerol, 5 mM KPO4, 200 mM KCl, 5 mM 2-mercaptoethanol, 0.05 mM EDTA and 200 mg ml/l BSA, pH 6.5). DNA construct reactions are prepared in 0.2 ml PCR tubes and cycled using the following conditions: 37 C for 5 or 15 min, 75 C for 20 min, −0.1 C/second to 60 C, then held at 60 C for 1 h. In general, a chew-back time of 5 min was used for overlaps less than 80 by and 15 min for overlaps greater than 80 bp. The base pairs used in the DNA construct assembly are generated from restriction digestion of DNA, synthetically synthesized DNA, and PCR products derived from plasmids and genomic DNA. All DNA base pairs have overlapping regions, which enable the assembly of the multiple DNA constructs into a single DNA construct. The DNA base pairs are integrated together in a linearized pcc1BAC, and thus the final assembly is a BAC able to replicate in a host cell.

The DNA construct is then introduced into an Escherichia coli host cell harboring the T7 RNA polymerase, such as BL21 and BL21 Lys. Isopropyl β-D-1-thiogalactopyranoside (IPTG) is used to induce the production of T7 RNA polymerase, which in turn, induces the expression of all genes on the metabolic pathway DNA construct under T7 RNA polymerase control. The metabolic pathway DNA construct can then be expressed to produce the steady state metabolic pathway enzymes encoded by a polynucleotide.

The desired 3-Hydroxypropionic acid product is determined by traditional analytical techniques as described herein.

3-Hydroxypropionic Acid Synthesis using a Steady State Metabolic Pathway with Hydrogen Symporter Transport of 3-Hydroxypropionic Acid: 3HP2BAC Design

The synthesis of 3-Hydroxypropionic acid from glucose in a steady state metabolic pathway in Escherichia coli is performed. In one embodiment, a steady state metabolic pathway in Escherichia coli for the synthesis of 3-Hydroxypropionic acid from glucose is identified. A constraint based model of Escherichia coli metabolism is used to determine a steady state metabolic pathway for the synthesis of 3-Hydroxypropionic acid from glucose in Escherichia coli using Escherichia coli model iAF1260 (Feist A M, et al, Mol Syst Biol. 2007;3:121.Feist). 3-Hydroxypropionic acid is not naturally produced in Escherichia coli and thus the following reactions identified using the KEG database are added to the Escherichia coli model: NADP-dependent glyceraldehyde-3-phosphate dehydrogenase from Clostridium acetobutylicum (GAPN(SEQ ID NO 69)), Alanine 2, 3, aminoaminase from US patent application US20100099143A1 (AAA(SEQ ID NO 47)), 2-hydroxy-3-oxopropionate reductase from Bacillus cereus G9842(MMSB(SEQ ID NO 48)), and alanine/pyruvate aminotransferase from pseudomonas aeruginosa (APTB(SEQ ID NO 49)). In addition a transport reaction is added to the iAF1260 model. For this example, it is assumed that 3-Hydroxypropionic acid is transported out of the Escherichia coli cell via a hydrogen symporter, (3-Hydroxypropionic acid[cytosol]+Hydrogen[cytosol]→3-Hydroxypropionic acid [paraplasm]+Hydrogen[paraplasm]), 3HP2t, which is added to the iAF1260 model. FBA is used to identify a steady state metabolic pathway by maximizing for 3-Hydroxypropionic acid, using glucose as a desired substrate. The glucose exchange reaction is set in FBA to allow the uptake of 1 mole of glucose/hour (M/h). The exchange reactions for 3-Hydroxypropionic acid, oxygen, water, and carbon dioxide, are set in FBA to allow the uptake and secretion of these metabolites to be unbounded.

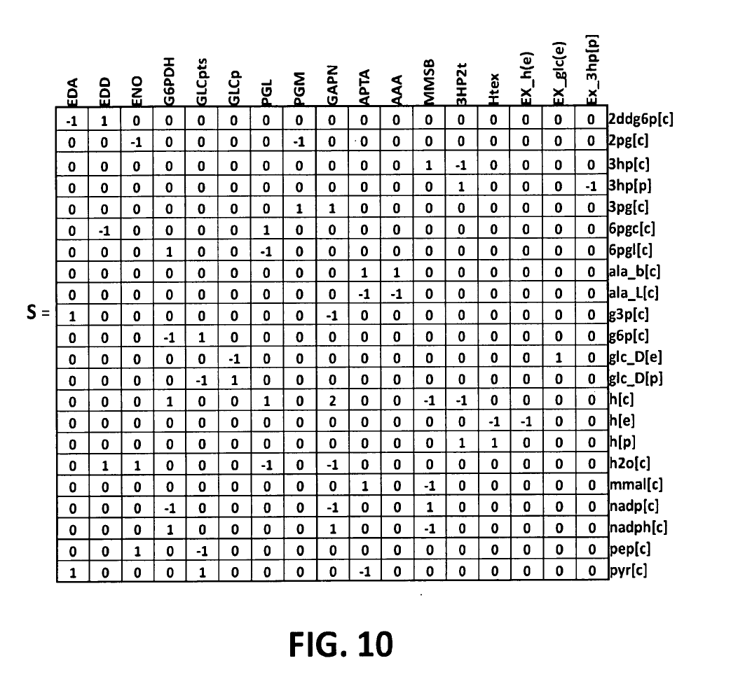

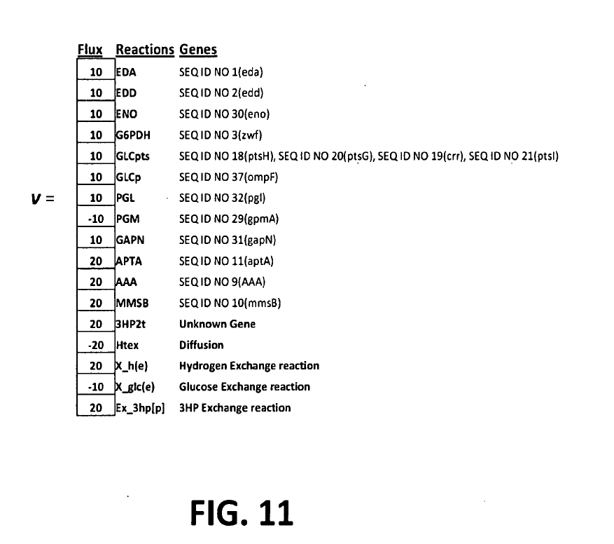

With added reactions to the iAF1260 model, there are many steady state metabolic pathways for the synthesis of 3-Hydroxypropionic acid, using glucose as a desired substrate. FIG. 9 shows one steady state metabolic pathway for the synthesis of 3-Hydroxypropionic acid, using glucose as a desired substrate, define as 3HP2BAC, having the reactions 2-keto-3-deoxygluconate 6-phosphate aldolase from Escherichia coli (EDA(SEQ ID NO 39)), phosphogluconate dehydratase from Escherichia coli (EDD(SEQ ID NO 40)), glucose 6-phosphate-1-dehydrogenase from Escherichia coli (G6P(SEQ ID NO 41)), glucose-specific PTS permease from Escherichia coli (GLCpts(PTSH(SEQ ID NO 56), CRR(SEQ ID NO 57), PTSG(SEQ ID NO 58), PTSI (SEQ ID NO 59))), 2,3-bisphosphoglycerate-dependent phosphoglycerate mutase from Escherichia coli (GPMA(SEQ ID NO 67)), enolase from Escherichia coli (ENO(SEQ ID NO 68)), NADP-dependent glyceraldehyde-3-phosphate dehydrogenase from Clostridium acetobutylicum (GAPN(SEQ ID NO 69)), 6-phosphogluconolactonase from Escherichia coli (PGL(SEQ ID NO 70)), and outer membrane porin F from Escherichia coli (OMPF(SEQ ID NO 75)). Alanine 2, 3, aminoaminase from US patent application US20100099143A1(AAA(SEQ ID NO 47)), 2-hydroxy-3-oxopropionate reductase from Bacillus cereus G9842(MMSB(SEQ ID NO 48)), and alanine/pyruvate aminotransferase from pseudomonas aeruginosa (APTB(SEQ ID NO 49)), Alanine 2, 3, aminoaminase from US patent application US20100099143A1(AAA(SEQ ID NO 47)), 2-hydroxy-3-oxopropionate reductase from Bacillus cereus G9842(MMSB(SEQ ID NO 48)), alanine/pyruvate aminotransferase from pseudomonas aeruginosa (APTB(SEQ ID NO 49)) and the 3HP2t transport reaction. For the synthesis of 3-Hydroxypropionic acid from glucose in Escherichia coli, stoichiometric matrix (S) and flux vector (v) and of the steady state metabolic pathway are shown in FIGS. 10 and 11, respectively demonstrating that S·v=0 and 3HP2BAC metabolic pathway is a steady state metabolic pathway.

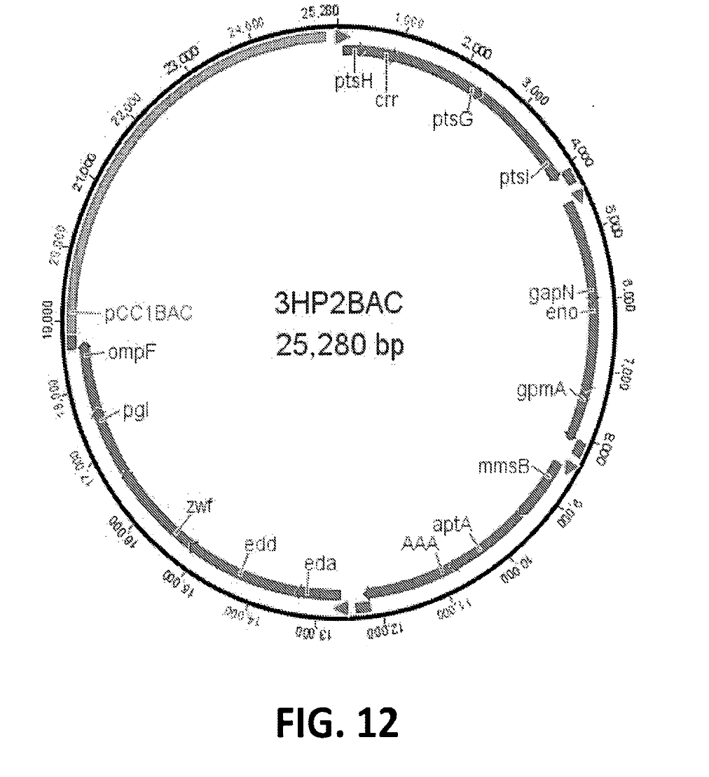

In one embodiment, the metabolic pathway DNA construct for the 3HP2BAC design, shown in FIG. 12, is created that has a sequence set forth in the following SEQ ID NOS: SEQ ID NO 1 (eda), SEQ ID NO 2 (edd), SEQ ID NO 30 (eno), SEQ ID NO 3 (zwf), SEQ ID NO 18 (ptsH), SEQ ID NO 20 (ptsG), SEQ ID NO 19 (crr), SEQ ID NO 21 (ptsl), SEQ ID NO 37 (ompF), SEQ ID NO 32 (pgl), SEQ ID NO 29 (gpmA), SEQ ID NO 31 (gapN), SEQ ID NO 11 (aptA), SEQ ID NO 9 (AAA), and SEQ ID NO 10 (mmsB).

Once a steady state metabolic pathway for the synthesis of 3-Hydroxypropionic acid from glucose has been identified, the enzymes of the steady state metabolic pathway are expressed in a host cell. A metabolic pathway DNA construct is created with each polynucleotide that encodes an enzyme of the 3HP1BAC steady state metabolic pathway. All enzymes are synthesized from a T7 RNA polymerase, thus allowing induction using Isopropyl β-D-1-thiogalactopyranoside (IPTG). A 4 chew-back, anneal and repair (CBAR) reaction buffer (20% PEG-8000, 600 mM Tris-HCl pH 7.5, 40 mM MgCl2, 40 mMDTT, 800 mM each of the four dNTPs and 4 mM NAD) is used for one-step thermocycled DNA assembly. DNA constructs are assembled in 40 ml reactions consisting of 10 ml 4 CBAR buffer, 0.35 ml of 4 U ml/l ExoIII (NEB), 4 ml of 40 U/ml Taq DNA ligase and 0.25 ml of 5 U/ml Ab-Taq polymerase. ExoIII is diluted 1:25 from 100 U ml/l in its stored buffer (50% glycerol, 5 mM KPO4, 200 mM KCl, 5 mM 2-mercaptoethanol, 0.05 mM EDTA and 200 mg ml/l BSA, pH 6.5). DNA construct reactions are prepared in 0.2 ml PCR tubes and cycled using the following conditions: 37 C for 5 or 15 min, 75 C for 20 min, −0.1 C/second to 60 C, then held at 60 C for 1 h. In general, a chew-back time of 5 min was used for overlaps less than 80 bp and 15 min for overlaps greater than 80 bp. The base pairs used in the DNA construct assembly are generated from restriction digestion of DNA, synthetically synthesized DNA, and PCR products derived from plasmids and genomic DNA. All DNA base pairs have overlapping regions, which enable the assembly of the multiple DNA constructs into a single DNA construct. The DNA base pairs are integrated together in a linearized pcc1BAC, and thus the final assembly is a BAC able to replicate in a host cell.

The DNA construct is then introduced into an Escherichia coli host cell harboring the T7 RNA polymerase, such as BL21 and BL21 Lys. Isopropyl β-D-1-thiogalactopyranoside (IPTG) is used to induce the production of T7 RNA polymerase, which in turn, induces the expression of all genes on the metabolic pathway DNA construct under T7 RNA polymerase control. The metabolic pathway DNA construct can then be expressed to produce the steady state metabolic pathway enzymes encoded by a polynucleotide.

The desired 3-Hydroxypropionic acid product is determined by traditional analytical techniques as described herein.

3-Hydroxypropionic Acid Synthesis using a Steady State Metabolic Pathway with Hydrogen Symporter Transport of 3-Hydroxypropionic Acid: 3HP3BAC Design

The synthesis of 3-Hydroxypropionic acid from glucose in a steady state metabolic pathway in Escherichia coli is performed. In one embodiment, a steady state metabolic pathway in Escherichia coli for the synthesis of 3-Hydroxypropionic acid from glucose is identified. A constraint based model of Escherichia coli metabolism is used to determine a steady state metabolic pathway for the synthesis of 3-Hydroxypropionic acid from glucose in Escherichia coli using Escherichia coli model iAF1260 (Feist A M, et al, Mol Syst Biol. 2007; 3:121.Feist). 3-Hydroxypropionic acid is not naturally produced in Escherichia coli and thus the following reactions identified using the KEG database are added to the Escherichia coli model: glycerol dehydratase from Klebsiella pneumonia (DHAB(DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)), NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)). In addition, a transport reaction is added to the iAF1260 model. For this example, it is assumed that 3-Hydroxypropionic acid is transported out of the Escherichia coli cell via a hydrogen symporter, (3-Hydroxypropionic acid[cytosol]+2 Hydrogen[cytosol]→3-Hydroxypropionic acid [paraplasm]+2 Hydrogen[paraplasm]), 3HP3t, which is added to the iAF1260 model. FBA is used to identify a steady state metabolic pathway by maximizing for 3-Hydroxypropionic acid, using glucose as a desired substrate. The glucose exchange reaction is set in FBA to allow the uptake of 1 mole of glucose/hour (M/h). The exchange reactions for 3-Hydroxypropionic acid, oxygen, water, and carbon dioxide, are set in FBA to allow the uptake and secretion of these metabolites to be unbounded.

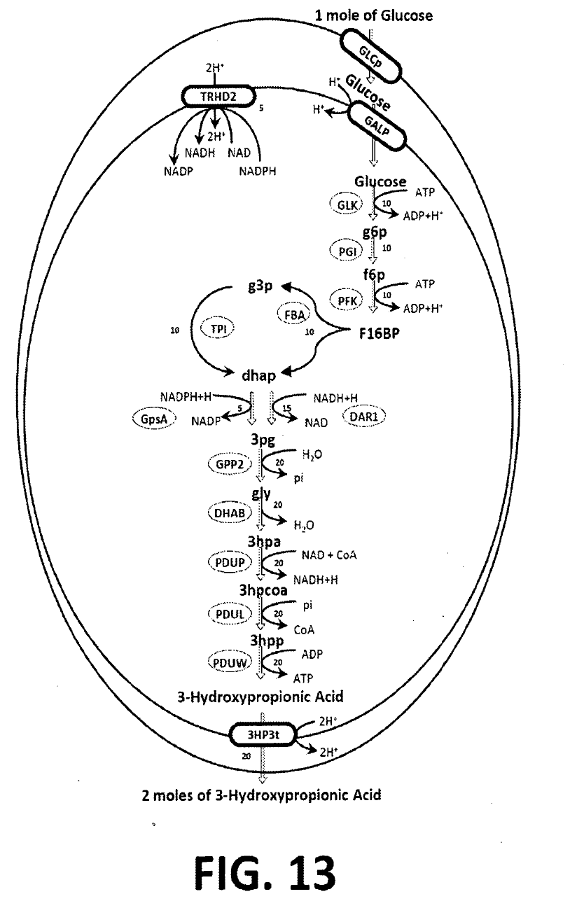

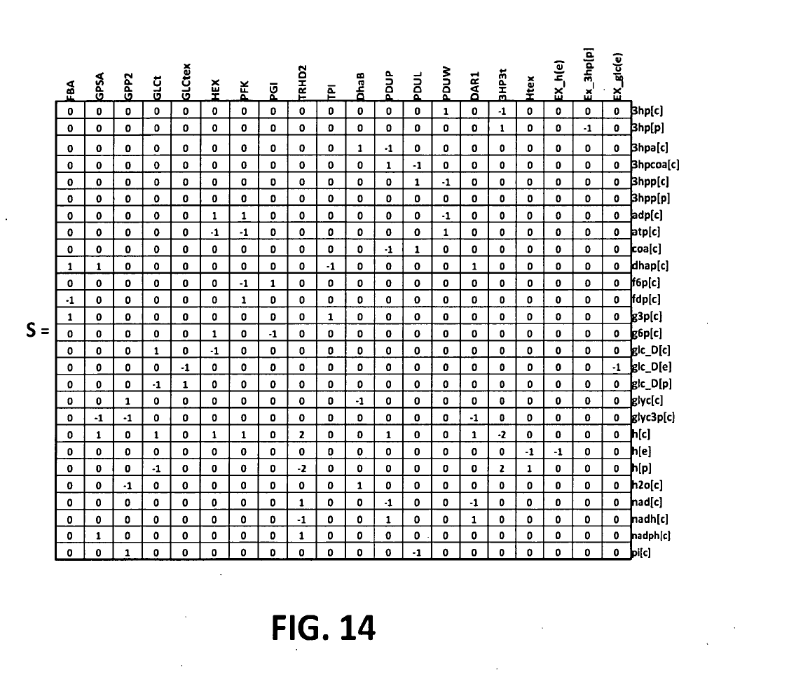

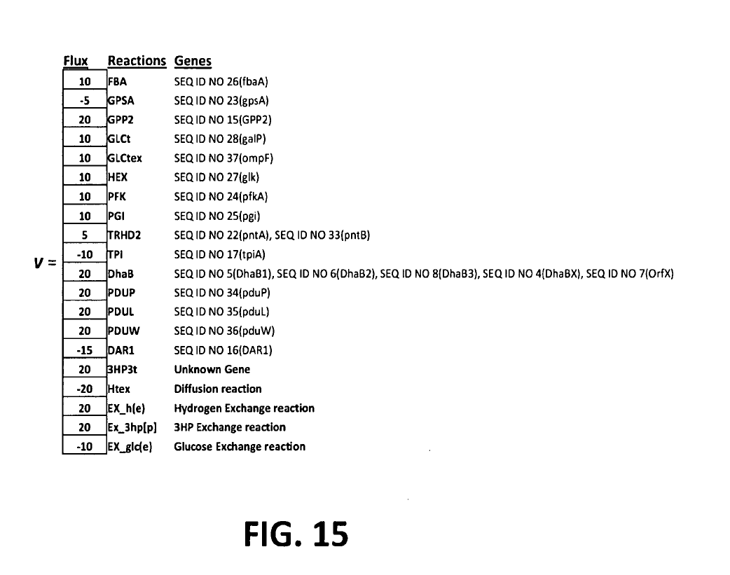

With added reactions to the iAF1260 model, there are many steady state metabolic pathways for the synthesis of 3-Hydroxypropionic acid, using glucose as a desired substrate. FIG. 13 shows one steady state metabolic pathway for the synthesis of 3-Hydroxypropionic acid, using glucose as a desired substrate, define as 3HP3BAC, having the reactions glycerol dehydratase from Klebsiella pneumonia (DHAB(DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)), NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)), triose phosphate isomerase from Escherichia coli (TPIA(SEQ ID NO 55)), glucokinase from Escherichia coli (GLK(SEQ ID NO 65)), galactose MFS transporter from Escherichia coli (GALP(SEQ ID NO 66)), 6-phosphofructokinase I from Escherichia coli (PFKA(SEQ ID NO 62)), phosphoglucose isomerase from Escherichia coli (PGI(SEQ ID NO 63)), fructose bisphosphate aldolase class II from Escherichia coli (FBAA(SEQ ID NO 64)), outer membrane porin F from Escherichia coli (OMPF(SEQ ID NO 75)), pyruvate kinase II from Escherichia coli (PYKA(SEQ ID NO 76)), pyridine nucleotide transhydrogenase from Escherichia coli (TRHD2(PNTA(SEQ ID NO 60), PNTB(SEQ ID NO 71))) and the 3HP3t transport reaction. For the synthesis of 3-Hydroxypropionic acid from glucose in Escherichia coli, stoichiometric matrix (S) and flux vector (v) and of the steady state metabolic pathway are shown in FIGS. 14 and 15, respectively demonstrating that S·v=0 and 3HP3BAC metabolic pathway is a steady state metabolic pathway.

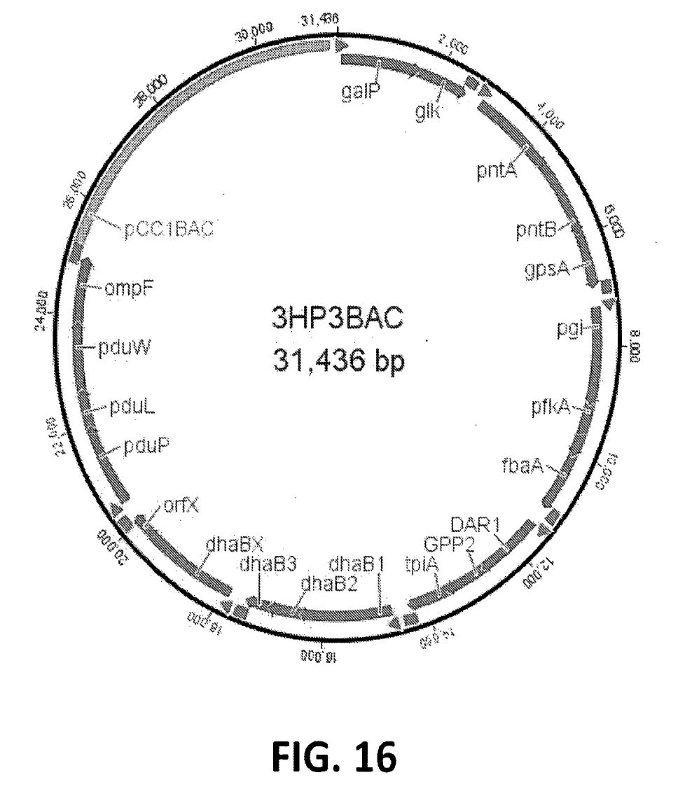

In one embodiment, the metabolic pathway DNA construct for the 3HP3BAC design, shown in FIG. 16, is created that has a sequence set forth in the following SEQ ID NOS: SEQ ID NO 26 (fbaA), SEQ ID NO 23 (gpsA), SEQ ID NO 15 (GPP2), SEQ ID NO 28 (galP), SEQ ID NO 37 (ompF), SEQ ID NO 27 (glk), SEQ ID NO 24 (pfkA), SEQ ID NO 25 (pgi), SEQ ID NO 22 (pntA), SEQ ID NO 33 (pntB), SEQ ID NO 17 (tpiA), SEQ ID NO 5 (DhaB1), SEQ ID NO 6 (DhaB2), SEQ ID NO 8 (DhaB3), SEQ ID NO 4 (DhaBX), SEQ ID NO 7 (OrfX), SEQ ID NO 34 (pduP), SEQ ID NO 35 (pduL), SEQ ID NO 36 (pduW), and SEQ ID NO 16 (DAR1).

Once a steady state metabolic pathway for the synthesis of 3-Hydroxypropionic acid from glucose has been identified, the enzymes of the steady state metabolic pathway are expressed in a host cell. A metabolic pathway DNA construct is created with each polynucleotide that encodes an enzyme of the 3HP1BAC steady state metabolic pathway. All enzymes are synthesized from a T7 RNA polymerase, thus allowing induction using Isopropyl β-D-1-thiogalactopyranoside (IPTG). A 4 chew-back, anneal and repair (CBAR) reaction buffer (20% PEG-8000, 600 mM Tris-HCl pH 7.5, 40 mM MgCl2, 40 mMDTT, 800 mM each of the four dNTPs and 4 mM NAD) is used for one-step thermocycled DNA assembly. DNA constructs are assembled in 40 ml reactions consisting of 10 ml 4 CBAR buffer, 0.35 ml of 4 U ml/l ExoIII (NEB), 4 ml of 40 U/ml Taq DNA ligase and 0.25 ml of 5 U/ml Ab-Taq polymerase. ExoIII is diluted 1:25 from 100 U ml/l in its stored buffer (50% glycerol, 5 mM KPO4, 200 mM KCl, 5 mM 2-mercaptoethanol, 0.05 mM EDTA and 200 mg ml/l BSA, pH 6.5). DNA construct reactions are prepared in 0.2 ml PCR tubes and cycled using the following conditions: 37 C for 5 or 15 min, 75 C for 20 min, −0.1 C/second to 60 C, then held at 60 C for 1 h. In general, a chew-back time of 5 min was used for overlaps less than 80 bp and 15 min for overlaps greater than 80 bp. The base pairs used in the DNA construct assembly are generated from restriction digestion of DNA, synthetically synthesized DNA, and PCR products derived from plasmids and genomic DNA. All DNA base pairs have overlapping regions, which enable the assembly of the multiple DNA constructs into a single DNA construct. The DNA base pairs are integrated together in a linearized pcc1BAC, and thus the final assembly is a BAC able to replicate in a host cell.

The DNA construct is then introduced into an Escherichia coli host cell harboring the T7 RNA polymerase, such as BL21 and BL21 Lys. Isopropyl β-D-1-thiogalactopyranoside (IPTG) is used to induce the production of T7 RNA polymerase, which in turn, induces the expression of all genes on the metabolic pathway DNA construct under T7 RNA polymerase control. The metabolic pathway DNA construct can then be expressed to produce the steady state metabolic pathway enzymes encoded by a polynucleotide.

The desired 3-Hydroxypropionic acid product is determined by traditional analytical techniques as described herein.

3-Hydroxypropionic Acid Synthesis using a Steady State Metabolic Pathway with Hydrogen Symporter Transport of 3-Hydroxypropionic Acid: 3HP4BAC Design

The synthesis of 3-Hydroxypropionic acid from glucose in a steady state metabolic pathway in Escherichia coli is performed. In one embodiment, a steady state metabolic pathway in Escherichia coli for the synthesis of 3-Hydroxypropionic acid from glucose is identified. A constraint based model of Escherichia coli metabolism is used to determine a steady state metabolic pathway for the synthesis of 3-Hydroxypropionic acid from glucose in Escherichia coli using Escherichia coli model iAF1260 (Feist A M, et al, Mol Syst Biol. 2007;3:121.Feist). 3-Hydroxypropionic acid is not naturally produced in Escherichia coli and thus the following reactions identified using the KEG database are added to the Escherichia coli model: NADP-dependent glyceraldehyde-3-phosphate dehydrogenase from Clostridium acetobutylicum (GAPN(SEQ ID NO 69)), Alanine 2, 3, aminoaminase from US patent application US20100099143A1 (AAA(SEQ ID NO 47)), 2-hydroxy-3-oxopropionate reductase from Bacillus cereus G9842(MMSB(SEQ ID NO 48)), alanine/pyruvate aminotransferase from pseudomonas aeruginosa (APTB(SEQ ID NO 49)), glycerol dehydratase from Klebsiella pneumonia (DHAB(DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)) , NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)). In addition, a transport reaction is added to the iAF1260 model. For this example, it is assumed that 3-Hydroxypropionic acid is transported out of the Escherichia coli cell via a hydrogen symporter, (3-Hydroxypropionic acid[cytosol]+2 Hydrogen[cytosol]→3-Hydroxypropionic acid [paraplasm]+2 Hydrogen[paraplasm]), 3HP3t, which is added to the iAF1260 model. FBA is used to identify a steady state metabolic pathway by maximizing for 3-Hydroxypropionic acid, using glucose as a desired substrate. The glucose exchange reaction is set in FBA to allow the uptake of 1 mole of glucose/hour (M/h). The exchange reactions for 3-Hydroxypropionic acid, oxygen, water, and carbon dioxide, are set in FBA to allow the uptake and secretion of these metabolites to be unbounded.

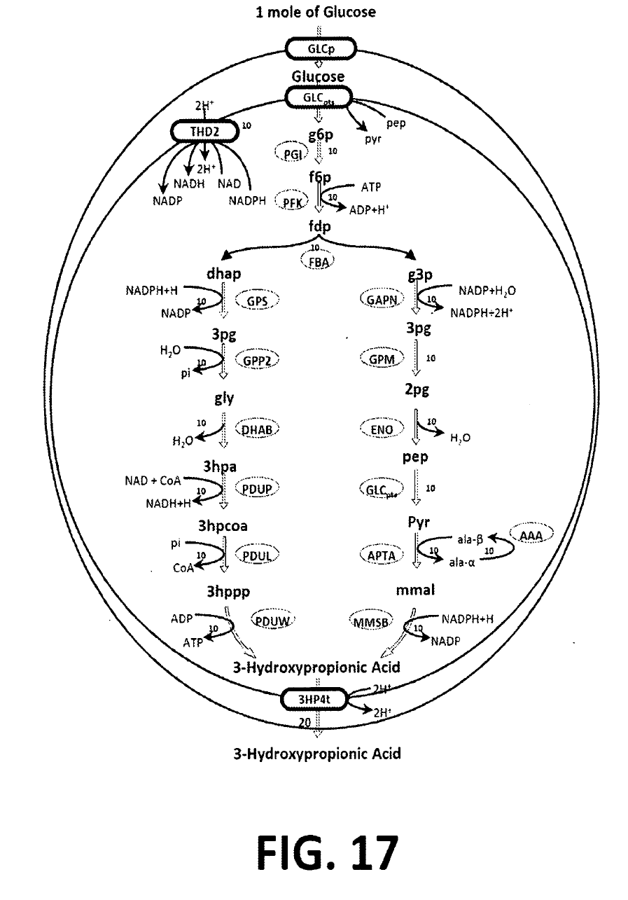

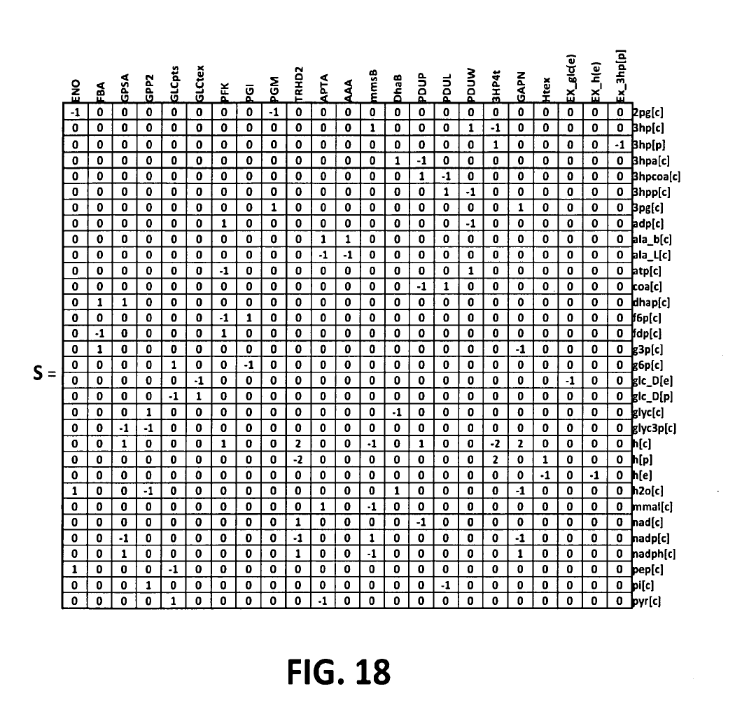

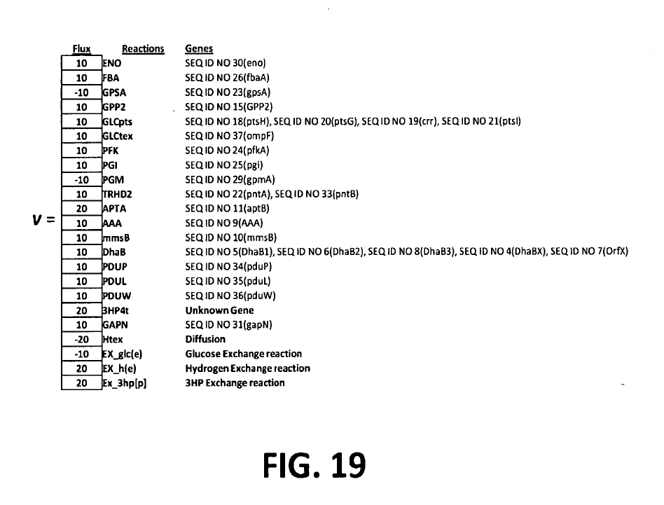

With added reactions to the iAF1260 model, there are many steady state metabolic pathways for the synthesis of 3-Hydroxypropionic acid, using glucose as a desired substrate. FIG. 17 shows one steady state metabolic pathway for the synthesis of 3-Hydroxypropionic acid, using glucose as a desired substrate, define as 3HP4BAC, having the reactions NADP-dependent glyceraldehyde-3-phosphate dehydrogenase from Clostridium acetobutylicum (GAPN(SEQ ID NO 69)), Alanine 2, 3, aminoaminase from US patent application US20100099143A1 (AAA(SEQ ID NO 47)), 2-hydroxy-3-oxopropionate reductase from Bacillus cereus G9842(MMSB(SEQ ID NO 48)), alanine/pyruvate aminotransferase from pseudomonas aeruginosa (APTB(SEQ ID NO 49)), glycerol dehydratase from Klebsiella pneumonia (DHAB(DHAB1(SEQ ID NO 43), DHAB2(SEQ ID NO 44), DHAB3(SEQ ID NO 46))), glycerol dehydratase reactivating factors from Klebsiella pneumonia (ORFX(SEQ ID NO 45), DHABX(SEQ ID NO 42)) , NAD-dependent glycerol-3-phosphate dehydrogenase from Saccharomyces cerevisiae (GPP2(SEQ ID NO 53)), DL-glycerol-3-phosphatase from Saccharomyces cerevisiae (DAR1(SEQ ID NO 54)), CoA-dependent propionaldehyde dehydrogenase from Salmonella enterica (PDUP(SEQ ID NO 72)), Phosphotransacylase from Salmonella enterica (PDUL(SEQ ID NO 73)), and propionate kinase from Salmonella enterica (PDUW(SEQ ID NO 74)), glucose-specific PTS permease from Escherichia coli (GLCpts(PTSH(SEQ ID NO 56), CRR(SEQ ID NO 57), PTSG(SEQ ID NO 58), PTSI (SEQ ID NO 59))), 6-phosphofructokinase I from Escherichia coli (PFKA(SEQ ID NO 62)), phosphoglucose isomerase from Escherichia coli (PGI(SEQ ID NO 63)), fructose bisphosphate aldolase class II from Escherichia coli (FBAA(SEQ ID NO 64)), outer membrane porin F from Escherichia coli (OMPF(SEQ ID NO 75)), pyruvate kinase II from Escherichia coli (PYKA(SEQ ID NO 76)), pyridine nucleotide transhydrogenase from Escherichia coli (TRHD2(PNTA(SEQ ID NO 60), PNTB(SEQ ID NO 71))), and the 3HP3t transport reaction. For the synthesis of 3-Hydroxypropionic acid from glucose in Escherichia coli, stoichiometric matrix (S) and flux vector (v) and of the steady state metabolic pathway are shown in FIGS. 18 and 19, respectively demonstrating that S·v=0 and 3HP4BAC metabolic pathway is a steady state metabolic pathway.

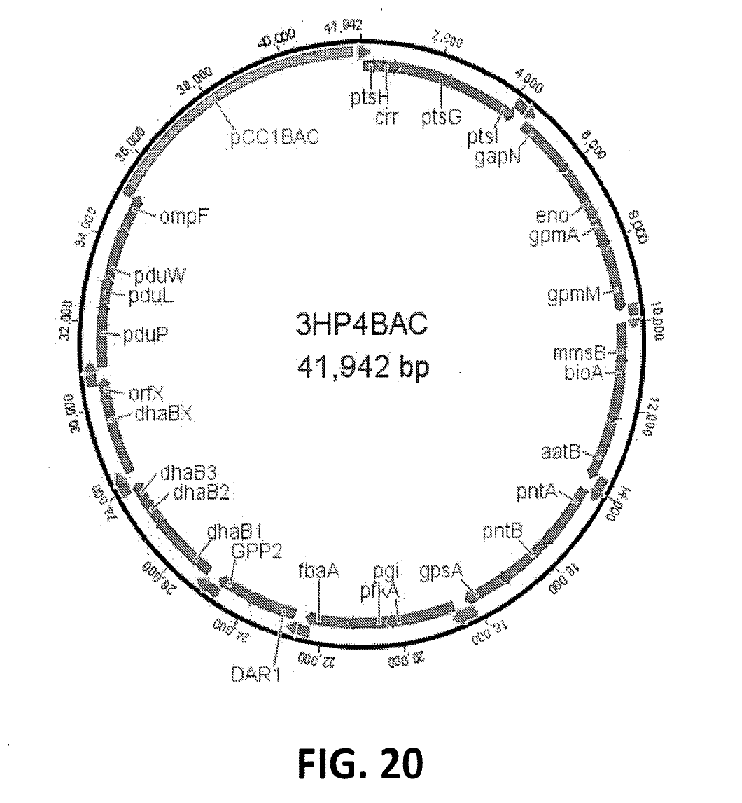

The metabolic pathway DNA construct for the 3HP4BAC design, shown in FIG. 20, is then created as that has a sequence set forth in the following SEQ ID NOS: SEQ ID NO 30 (eno), SEQ ID NO 26 (fbaA), SEQ ID NO 23 (gpsA), SEQ ID NO 15 (GPP2), SEQ ID NO 18 (ptsH), SEQ ID NO 20 (ptsG), SEQ ID NO 19 (crr), SEQ ID NO 21 (ptsl), SEQ ID NO 37 (ompF), SEQ ID NO 24 (pfkA), SEQ ID NO 25 (pgi), SEQ ID NO 29 (gpmA), SEQ ID NO 22 (pntA), SEQ ID NO 33 (pntB), SEQ ID NO 11 (aptB), SEQ ID NO 9 (AAA), SEQ ID NO 10 (mmsB), SEQ ID NO 5 (DhaB1), SEQ ID NO 6 (DhaB2), SEQ ID NO 8 (DhaB3), SEQ ID NO 4 (DhaBX), SEQ ID NO 7 (OrfX), SEQ ID NO 34 (pduP), SEQ ID NO 35 (pduL), SEQ ID NO 36 (pduW), and SEQ ID NO 31 (gapN).

Once a steady state metabolic pathway for the synthesis of 3-Hydroxypropionic acid from glucose has been identified, the enzymes of the steady state metabolic pathway are expressed in a host cell. A metabolic pathway DNA construct is created with each polynucleotide that encodes an enzyme of the 3HP1BAC steady state metabolic pathway. All enzymes are synthesized from a T7 RNA polymerase, thus allowing induction using Isopropyl β-D-1-thiogalactopyranoside (IPTG). A 4 chew-back, anneal and repair (CBAR) reaction buffer (20% PEG-8000, 600 mM Tris-HCl pH 7.5, 40 mM MgCl2, 40 mMDTT, 800 mM each of the four dNTPs and 4 mM NAD) is used for one-step thermocycled DNA assembly. DNA constructs are assembled in 40 ml reactions consisting of 10 ml 4 CBAR buffer, 0.35 ml of 4 U ml/l ExoIII (NEB), 4 ml of 40 U/ml Taq DNA ligase and 0.25 ml of 5 U/ml Ab-Taq polymerase. ExoIII is diluted 1:25 from 100 U ml/l in its stored buffer (50% glycerol, 5 mM KPO4, 200 mM KCl, 5 mM 2-mercaptoethanol, 0.05 mM EDTA and 200 mg ml/l BSA, pH 6.5). DNA construct reactions are prepared in 0.2 ml PCR tubes and cycled using the following conditions: 37 C for 5 or 15 min, 75 C for 20 min, −0.1 C/second to 60 C, then held at 60 C for 1 h. In general, a chew-back time of 5 min was used for overlaps less than 80 bp and 15 min for overlaps greater than 80 bp. The base pairs used in the DNA construct assembly are generated from restriction digestion of DNA, synthetically synthesized DNA, and PCR products derived from plasmids and genomic DNA. All DNA base pairs have overlapping regions, which enable the assembly of the multiple DNA constructs into a single DNA construct. The DNA base pairs are integrated together in a linearized pcc1BAC, and thus the final assembly is a BAC able to replicate in a host cell. The DNA construct is then introduced into an Escherichia coli host cell harboring the T7 RNA polymerase, such as BL21 and BL21 Lys. Isopropyl β-D-1-thiogalactopyranoside (IPTG) is used to induce the production of T7 RNA polymerase, which in turn, induces the expression of all genes on the metabolic pathway DNA construct under T7 RNA polymerase control. The metabolic pathway DNA construct can then be expressed to produce the steady state metabolic pathway enzymes encoded by a polynucleotide.

The desired 3-Hydroxypropionic acid product is determined by traditional analytical techniques as described herein.

The foregoing has described the principles, embodiments, and modes of operation of the present disclosure. However, the present disclosure should not be construed as being limited to the particular embodiments described above, as they should be regarded as being illustrative and not as restrictive. It should be appreciated that variations may be made in those embodiments by those skilled in the art without departing from the scope of the present disclosure.

Modifications and variations of the present disclosure are possible in light of the above teachings. It is therefore to be understood that the present disclosure may be practiced otherwise than as specifically described herein.

What is claimed is:

1. A method for increasing the production of a desired product, comprising:
identifying a steady state metabolic pathway for the synthesis of a desired product from a desired substrate;
producing a polynucleotide encoding one or more polypeptide that participates in the steady state metabolic pathway for the synthesis of the desired product from the desired substrate;
introducing the polynucleotide encoding a polypeptide into a host cell; transforming a host cell with an expression vector comprising an expressible polynucleotide encoding a polypeptide; and
cultivating the host cell under a culture condition that induces the production of the desired product.

2. The method of claim 1, further comprising collecting the desired product from the host cell.

3. The method of claim 1, wherein the desired product is glucose.

4. The method of claim 1, wherein the desired substrate is 3-Hydroxypropionic acid.

5. The method of claim 1, wherein the host cell is Escherichia coli.

6. The method of claim 1, wherein the host cell comprises a polynucleotide for T7 RNA polymerase.

7. The method of claim 1, wherein the one or more polypeptides have a sequence selected from the group consisting of SEQ ID NO: 39, 40, 41, 50, 51, 56, 57, 58, 59, 67, 68, 69, 70, and 75.

8. The method of claim 1, wherein the one or more polypeptides have a sequence selected from the group consisting of SEQ ID NO: 44, 46, 45, 42, 53, 54, 72, 73, 74, 55, 56, 57, 58, 59, 62, 63, 64, 75, and 76.

9. The method of claim 1, wherein the one or more polypeptides have a sequence selected from the group consisting of SEQ ID NO: 39, 40, 41, 56, 57, 58, 59, 67, 68, 69, 70, 75, 47, 48, and 49.

10. The method of claim 1, wherein the one or more polypeptides have a sequence selected from the group consisting of SEQ ID NO: 43, 44, 46, 45, 42, 53, 54, 72, 73, 74, 55, 65, 66, 62, 63, 64, 75, 76, 60, and 71.

11. The method of claim 1, wherein the one or more polypeptides have a sequence selected from the group consisting of SEQ ID NO: 42, 43, 44, 45, 46, 47, 48, 49, 53, 56, 57, 58, 59, 60, 61, 62, 63, 64, 67, 68, 69, 71, 72, 73, 74, and 75.

12. The method of claim 1, wherein the expression vector comprises a promoter operably linked to the polynucleotide.

13. The method of claim 1, wherein the polynucleotide encoding the expressible polynucleotide comprises the polynucleotide selected from the group consisting of SEQ ID NO: 37, 18, 20, 19, 21, 3, 32, 1, 2, 30, 31, 29, 12, 14, and 13.

14. The method of claim 1, wherein the polynucleotide encoding the expressible polynucleotide comprises the polynucleotide selected from the group consisting of SEQ ID NO: 6, 8, 7, 4, 15, 16, 34, 35, 36, 17, 18, 19, 20, 21, 24, 25, 26, 37, and 38.

15. The method of claim 1, wherein the polynucleotide encoding the expressible polynucleotide comprises the polynucleotide selected from the group consisting of SEQ ID NO: 1, 2, 3, 18, 19, 20, 21, 29, 30, 31, 32, 37, 9, 10, and 11.

16. The method of claim 1, wherein the polynucleotide encoding the expressible polynucleotide comprises the polynucleotide selected from the group consisting of SEQ ID NO: 5, 6, 8, 7, 4, 15, 16, 34, 35, 36, 17, 27, 28, 24, 25, 26, 37, 38, 22, and 33.

17. The method of claim 1, wherein the polynucleotide encoding the expressible polynucleotide comprises the polynucleotide selected from the group consisting of SEQ ID NO: 4, 5, 6, 7, 8, 9, 10, 11, 15, 18, 19, 20, 21, 22, 23, 24, 25, 26, 29, 30, 31, 33, 34, 35, 36, and 37.

18. A method for increasing the production of a desired product, comprising:
identifying a steady state metabolic pathway for the synthesis of a desired product from a desired substrate;
producing a polynucleotide with nucleic acid sequences encoding all polypeptides that participate in the steady state metabolic pathway for the synthesis of the desired product from the desired substrate;
introducing the polynucleotide encoding a polypeptide into a host cell;
expressing the polynucleotides encoding all polypeptides of the steady state metabolic pathway; and
cultivating the host cell under a culture condition that induces the production of the desired product.

19. The method of claim 1, wherein one or more nucleic acid sequence encoding a polypeptide that participates in the steady state metabolic pathway is not incorporated into the polynucleotide.

20. A method for increasing the production of a desired product, comprising:
identifying a steady state metabolic pathway for the synthesis of a desired product from a desired substrate; and
expressing all polypeptides of the steady state metabolic pathway within a host cell.
